(19) United States
(12) Patent Application Publication
(10) Pub. No.: US 2026/0309725 A1
(43) Pub. Date: Oct. 8, 2026

(54) A NASAL FLUID SAMPLE COMPRISING ABETA, PTAU AND/OR TTAU

(71) Applicant: Noselab GmbH, München (DE)

(72) Inventors: Mareike Albert, München (DE); Thomas Heydler, Gmund (DE); Hilary Wunderlich, Kiefersfelden (DE); Klaus Peter Kaspar, Weilheim (DE); Klaus Hallermeyer, Feldafing (DE); Arno Schäpe, Starnberg (DE); Richard Metzler, München (DE); Harald Waltenberger, Gilching (DE); Marion San Nicoló, Pullach im Isartal (DE); Sabine Mertzig, Dietersheim (DE)

(21) Appl. No.: 19/483,772

(22) PCT Filed: May 10, 2024
(86) PCT No.: PCT/EP2024/063000

§ 371 Date: Nov. 12, 2025

(30) Foreign Application Priority Data
May 12, 2023 (EP) 23173258.7
Jul. 6, 2023 (EP) 23184006.7
Oct. 23, 2023 (EP) 23205373.6

(51) Int. Cl. G01N 33/68

(52) U.S. Cl. CPC G01N 33/6896; G01N 2333/4709; G01N 2800/2821

(57) ABSTRACT

The invention relates to a nasal fluid sample obtained from a subject comprising the marker protein(s) β amyloid (Aβ), phosphorylated Tau (pTau) and/or total Tau (tTau). The invention further relates to a nasal fluid sample comprising the marker protein(s) Aβ, pTau and/or tTau for use in a method for the aid in diagnosis of neurodegenerative diseases and the use of a nasal fluid sample comprising the marker protein(s) Aβ, pTau and/or tTau for the aid in diagnosis of a neurodegenerative disease. The invention further relates to a method for the aid in diagnosis of a neurodegenerative disease in a subject/individual.

The invention relates to a nasal fluid sample obtained from a subject comprising the marker protein(s) beta amyloid (Abeta, (Aβ)), phosphorylated Tau (pTau) and/or total Tau (tTau). The invention further relates to a nasal fluid sample comprising the marker protein(s) Aβ, pTau and/or tTau for use in a method for the aid in diagnosis of neurodegenerative diseases and the use of a nasal fluid sample comprising the marker protein(s) Aβ, pTau and/or tTau for the aid in diagnosis of a neurodegenerative disease. The invention further relates to a method for the aid in diagnosis of a neurodegenerative disease in a subject/individual.

### BACKGROUND OF THE INVENTION

Neurodegenerative diseases represent a major threat to the human health. These diseases are becoming increasingly prevalent, in part because of the increase of the elderly population in recent years. Some examples of neurodegenerative diseases are Alzheimer's disease (AD), Parkinson's disease (PD), Chronic Traumatic Encephalopathy (CTE), Creutzfeldt-Jakob's disease (CJD), Dementia with Lewy bodies, Vascular Dementia (VD), Huntington's Disease (HD), Frontotemporal Dementia (FTD), REM sleep behaviour disorder, Multiple system atrophy (MSA), Amyotrophic lateral sclerosis (ALS) and Multiple sclerosis (MS).

Alzheimer's disease (AD), as an example of a neurodegenerative disease (ND), is the most common form of dementia, accounting for 60-80% of all dementia syndromes (see ‘2020 Alzheimer's disease facts and figures’, Alzheimers Dement (2020), 16(3), pp. 391-460, doi: 10.1002/ALZ.12068). The “A/T/(N)” research framework introduced by the National Institute on Aging and Alzheimer's Association (NIA-AA) in 2018 (see Jack et al., Alzheimer's and Dementia, (2018), 14(4), pp. 535-562, doi: 10.1016/j.jalz.2018.02.018) comprises several biological biomarkers for the diagnosis of AD, including: low levels of cerebrospinal fluid (CSF) Aβ(1-42) (Aβ42) or a low CSF Aβ(1-42)/Aβ(1-40) ratio as a marker of Aβ pathology (labeled “A”); elevated CSF tau phosphorylated at threonine 181 (pTau181) as a marker of fibrillar tau (labeled “T”);
and elevated CSF total tau (tTau) as marker of neurodegeneration or neuronal injury (labeled “(N)”). The presence of different markers determines each subject's individual disease status, i.e., whether the patient only exhibits pathological changes of AD (Aβ pathology: A+) or has AD (Aβ and fibrillar tau pathology: A+T+); see Jack et al., Alzheimer's and Dementia, (2018), 14(4), pp. 535-562, doi: 10.1016/j.jalz.2018.02.018.

Currently, marker proteins for A-, T- and N-based diagnosis are obtained by either lumbar puncture and the measurement of CSF or by positron emission tomography (PET) (Hansson et al., Alzheimers Dement (2018), 14(11), pp. 1470-1481, doi: 10.1016/J.JALZ.2018.01.010; Pannee et al., J Neurochem (2016), 139(4), pp. 651-658, doi: 10.1111/JNC.13838; Bittner et al., Alzheimers Dement (2016), 12(5), pp. 517-526, doi: 10.1016/J.JALZ.2015.09.009; Alcolea et al., Ann Clin Transl Neurol (2019), 6(9), pp. 1815-1824, doi: 10.1002/ACN3.50873). Subjects with preclinical AD show changes in CSF amyloid and tau levels/biomarkers. Specifically, a decreased level of Aβ42 and increased level of total Tau (tTau) and phosphorylated Tau (pTau), particularly pTau181 in CSF are useful for the early diagnosis of AD (Müller et al., PLoS One. (2019), 14(8): e0221365).

CSF biomarkers may have several advantages over PET. First, CSF tests are less expensive and have no radiation exposure issues compared to PET. Second, prior studies have shown that changes in CSF Aβ42 precede PET uptake (Jack et al., Lancet Neurol. (2013); 12(2), pp. 207-216; Bateman et al., N Engl J Med. (2012), 367(9), pp. 795-804) suggesting that CSF biomarkers may be more sensitive in identifying subjects with preclinical AD. Third, the most noteworthy benefit of CSF biomarker analysis is that CSF provides more information about AD-related pathologies than PET. That is, CSF samples do not only yield information on the marker protein Aβ, but also on the Tau pathology biomarker, phosphorylated Tau (p-Tau), and the neurodegenerative marker, total tau (t-Tau) (Apostolova et al., Neurobiol Aging. (2010), 31(8), pp. 1284-1303).

In principle, after the analysis of the marker proteins in CSF, an evaluation of these marker proteins in CSF is always based on a summary-evaluation of all individual markers to form an integrated overall result, so that a reliable and diagnostically valuable statement can be achieved. On one hand, this diagnostic strategy allows the recognition of typical patterns of marker proteins and on the other hand, it enables plausibility checks to avoid analytical errors (see Guideline for Diagnosis and Therapy in Neurology: Tumani H., Petereit H.-F. et al., Lumbalpunktion und Liquordiagnostik, S1-Leitlinie, 2019, in: Deutsche Gesellschaft für Neurologie (Hrsg.), Leitlinien für Diagnostik und Therapie in der Neurologie).

The summary-evaluation of the marker proteins Aβ42, Aβ40, pTau181 and tTau reveals a typical pattern for these proteins in CSF. From numerous examinations of CSF in patients with a neurodegenerative disorder it is well known that these marker proteins do not change independently of each other and rather show a distinguished, very strongly correlated change. It is known, for example, that pTau181, a phosphorylated form of tTau, and tTau nearly linearly increase together in neurodegenerative processes. They also inversely correlate to the Aβ42/Aβ40 ratio—lower Aβ42/Aβ40 ratios are strongly correlated to elevated pTau181 and tTau levels in CSF. These correlations are all strong and highly significant and thus can be seen as a pattern to form the integrated overall result, that can diagnostically be interpreted as high likelihood for the presence of an AD pathology and additionally analytical errors can be indirectly excluded.

The olfactory system is involved early in the context of neurodegenerative diseases. Olfactory dysfunctions are among the earliest clinical prodromal symptoms in ND diseases. A few studies were able to detect single AD Biomarker's in nasal samples, like nasal biopsies and or nasal lavages for example. There were strong inconsistencies between the findings of the studies. Sometimes e.g. increased concentration of Aβ42 could be reported in the context of clinically diagnosed AD cases and in other studies decreased concentrations were found. Other studies could detect Aβ only after vigorous pre-treatment of the nasal samples with denaturing substances. Some studies reported increased concentrations of pTau in clinically proven AD cases that could never be correlated with any biomarker proven AD and or ND diseases. All of the previous working groups dealt with the problem that quantities and or concentrations of marker proteins were significantly low in nasal samples, in nearly 50% of the cases below the lower limit of quantification and detection and they additionally reported high variability, therefore interpretation of data was heavily limited (see Kim et al., Sci Rep (2019), 9(1), p. 4966, doi: 10.1038/s41598-019-41429-1). However, as only single proteins of the brain could be detected a consistent pattern of marker proteins of neurodegenerative diseases such as AD could not be determined and none of the previous studies reported CSF-like quantities or CSF-like quaternary structures. The cribriform plate (CP) of the anterior skull base is a thin bony structure that forms the roof of the nasal cavity. It embraces the olfactory bulb (OB), an elongation of the central nervous system (CNS). Deriving from the OB, millions of olfactory nerve fibers pervade the roof of the nasal cavity through the CP. This large area (about 23 cm²), where the CNS is communicating with the nose, is also defined as the Brain Nose Interface (BNI). Further, it has been reported that the BNI also can be seen as physiological connection between the brain and the lymphatic system of the nose, constituting a drainage route for CSF and its metabolites along the olfactory route into the lymphatic system of the nose and from there into the retropharyngeal and cervical lymph nodes.

The applicant reported an application device for positioning an absorptive matrix (AM)-element within the BNI to obtain nasal fluid sample from subjects, particularly healthy subjects or subjects suffering from subjective or objective cognitive decline that can be due to neurodegenerative diseases such as AD; EP 4 000 534 A1 and WO 2022/101311. This nasal secretion collection device is a tool for the standardized collection of nasal secretion from the vicinity of the olfactory cleft and combines the requirement of extracting sufficient high-quality volume of the nasal fluid sample from this location while also avoiding artifacts in the subsequent analysis of the collected nasal fluid samples due to interferences with, e.g., blood or cell debris or nasal fluids form other areas of the nose. Moreover, the nasal mucosa in the vicinity of the olfactory cleft should be handled with caution and not exposed to any injury.

In the present application, it has unprecedentedly been demonstrated that marker proteins that are associated with neurodegenerative diseases, particularly but not limited to AD, PD and CTE, can be detected in nasal fluid samples obtained from subjects, preferably as collected from the vicinity of the olfactory cleft region and more preferably with the applicant's reported application device, without alteration of the CSF-like pattern of the marker proteins. This finding was surprising and unexpected because the skilled person would not have expected that the nasal fluid samples can be used like CSF samples obtained by lumbar puncture, i.e., the nasal fluid samples can be used as an alternative fluid sample that allows the in vitro diagnosis of neurodegenerative diseases such as AD, PD and CTE. Further, the fact the nasal fluid samples can be equally evaluated on a summary-evaluation of all individual markers to form an integrated overall result to allow a reliable and diagnostically valuable statement was also unexpected. Thus, the nasal fluid samples allow the same diagnostic strategy to interpret the marker protein(s) to identify typical signatures of the marker protein(s) and provide plausibility checks to avoid analytical errors. In particular, the claimed invention is unexpected and surprising because only limited knowledge exists about the peripheral outflow system of CSF across the brain nose interface. In a few mammalian studies and postmortem studies on human cadavers it was reported that direct routes exist that drain CSF into a lymphatic network located in the nasal submucosa of the nose and from there on into retropharyngeal and cervical lymph nodes (see Spera et al., eBioMedicine (2023), 91: 104558 (Published Online: https://doi.org/10.1016/j.ebiom.2023.104558). However, the exact anatomical and physiological mechanisms for this drainage system are still not known. In addition, the information such as the volume of CSF drainage into the lymphatic system over time or the marker protein patterns of drained CSF are also unknown. Further, possible interferences or alterations of the marker proteins after the drainage still remained unclear. Given the fact that the previously described drainage routes are described to drain the CSF across the BNI directly into lymphatic vessels, the skilled person would not have expected that without having access to the lymphatic system, which is covered by mucosal tissue, it would be possible to obtain nasal sample fluids that provide these CSF-like information e.g. on the biomarker proteins Aβ, pTau and tTau. Moreover, it was totally unexpected for the skilled person to find the nearly identical pattern or inter-protein correlations between the marker protein(s), nor would the skilled person have expected to find nearly identical distributions of the different oligomeric protein species of each marker protein or equal or even higher amounts of these marker proteins in a nasal fluid sample.

Accordingly, the problem to be solved by the present application resides on the identification of an alternative biological sample that could yield a comparable diagnosis of neurodegenerative diseases such as AD, PD and CTE.

### SUMMARY OF THE INVENTION

This technical problem is solved by the present invention by providing the embodiments as defined in the claims. In particular, the invention provides a nasal fluid sample obtained from a subject comprising the marker proteins beta amyloid (Abeta/Aβ), phosphorylated Tau (p-Tau/pTau) and/or total Tau (t-Tau/tTau). The invention further provides said nasal fluid sample for use in a method of diagnosis of neurodegenerative diseases and the use of said nasal fluid sample for the diagnosis of a neurodegenerative disease. The invention further relates to a method for the aid in diagnosis of a neurodegenerative disease in a subject.

In the appended Examples, the inventors unexpectedly and surprisingly demonstrated that the nasal fluid sample obtained from a subject according to the invention allows the same diagnosis as an analysis of CSF of the same subject by analysis of marker proteins associated with neurodegenerative disease. It has been surprisingly and unexpectedly found that the nasal fluid sample according to the invention comprises a nearly identical marker protein signature and/or protein correlations as also seen in CSF samples. Further, it was also totally unexpected that the distribution of the different oligomeric protein species of the marker protein(s) is (are) nearly identical in the nasal fluid samples and corresponding CSF samples from the same individual. Also, the fact that the concentration of the marker protein(s) in the nasal fluid are equivalent or even higher than in corresponding CSF samples was unexpected.

It has been demonstrated for the first time that by analyzing a nasal fluid sample it is possible to quantify the same marker protein(s) as in corresponding CSF samples. It further has been demonstrated for the first time, that the quantified marker protein(s) follow(s) the same pattern in nasal fluids as in CSF when evaluated in a summary-evaluation in the sense of an integrated overall result. This means that it has been demonstrated for the first time that, e.g., the Aβ42/Aβ40 ratio is inversely correlated to the tau markers. Likewise, it has been demonstrated that the two tau isoforms (pTau and tTau) show the same strong nearly linear correlation towards each other, meaning that whenever pTau is elevated tTau is also elevated. So, for the first time it has been demonstrated that in a nasal fluid sample the same pattern can be used to provide a plausibility check to avoid analytical errors and the nasal fluid can be used to identify pattern or correlation of marker proteins that are typical for the presence of a neurodegenerative disease in a subject (e.g. low Aβ42/Aβ40 ratios with high pTau and tTau levels are associated with AD). Additionally, by comparing the two corresponding samples—the nasal fluid sample and the CSF sample—from the same subject, it has been also demonstrated that the distribution (the different sizes and quantities) of monomers and oligomers, also show a nearly identical pattern. Thus, the pattern of the oligomers of the nasal sample is representative of that in the CSF sample from the same subject. Therefore, analyzing the nasal fluid sample(s) from subjects regarding the relative protein amount and by quantifying different oligomeric species can allow identification of subject with different neurodegenerative diseases. E.g., subjects with amyloid pathology (A+) or without Amyloid pathology (A−) show different oligomeric structures and relative amounts of the marker protein(s) when comparing the nasal fluid samples of these individuals. This has never been demonstrated before, and since it was surprising to find CSF in the first place, so to be able to measure or analyze a nasal fluid sample with equal or even higher amounts of the marker protein(s) and to be able to analyze their nearly identical pattern as well as oligomeric structures was surprising and not expected at all.

### DESCRIPTION OF THE FIGURES

The Figures show:

FIG. 1: Color Scale to assess blood content of the nasal secretion eluate.

FIG. 2: Aβ40: different quantities of oligomers can be detected at 56 kDa and 100 kDa by Simple Western™ Jess using an Aβ40-specific antibody from R and D.

FIG. 3: Aβ40: An example of a classification of CSF-verified A− and A+ individuals by the 56 kDa band. A+ subjects have less of the 56 kDa Aβ40 oligomer than A− subjects using an Aβ40-specific antibody from R and D.

FIG. 4: Aβ40: Lower molecular weight oligomers and monomers can be detected by Simple Western™ Jess using an Aβ40-specific antibody from R and D. A− subjects have a higher amount of monomeric Aβ-40.

FIG. 5: Aβ40: A− subjects have more monomers at <12 kDa than A+ as shown in this example plot from two subjects using an Aβ40-specific antibody from R and D.

FIG. 6: Aβ42: A+ have decreased total levels of Aβ42 and an altered ratio of oligomeric bands at 56 and 32 kDa as exemplified by one A+ and one A− subject using an Aβ42-specific antibody from Fujifilm Wako.

FIG. 7: pan-Aβ antibody recognizes multiple oligomeric Aβ structures in nasal secretion using the 6E10 antibody.

FIG. 8: detected tTau levels are increased in N+ relative to N− as shown in this example of an N+ and an N− subject using the Tau specific antibody from Biolegend.

FIG. 9: pTau181 levels are decreased in T+ relative to T− as demonstrated in this example of one T+ and one T− patient sample using the pTau specific antibody from Cell Signaling.

FIG. 10: Aβ40: directly comparing the recognized Aβ40 structures in a nasal fluid sample and a CSF sample from the same individual/subject reveals nearly identical structures at approximately 62 kDa and approximately 231 kDa using the Aβ40 specific antibody from BioLegend (purified (azide-free) anti-β-Amyloid, 1-40 (11A50-B10), BioLegend, cat. #805409). It was 6 times more CSF sample volume applied, indicating that the amount of structures at approximately 62 kDa and approximately 231 kDa is at least comparable to CSF or higher than CSF in the nasal secretion sample. Additional structures can be found in the nasal secretion sample at approximately 20 kDa, 32 kDa, and 98 kDa.

FIG. 11: Aβ42: directly comparing the recognized Aβ42 structures in a nasal secretion sample and a CSF sample from the same individual reveals structures in the nasal secretion sample at approximately 33 kDa, 56 kDa, 75 kDa, and 99 kDa using the Aβ42 specific antibody from Fujifilm Wako (Anti Amyloid β42(43), Monoclonal Antibody (BC05), Fujifilm Wako, cat. #010-26903). These structures cannot be seen in CSF even when 6 times more CSF sample volume was applied. This is indicating that the amounts of the structures seen in the nasal secretion sample are higher than in the corresponding CSF sample.

FIG. 12: tTau: directly comparing the recognized tTau structures in a nasal secretion sample and a CSF sample from the same individual reveals nearly identical structures at 63 kDa using the tTau specific antibody from BioLegend (Purified anti-Tau, 404-441 Antibody (mouse), BioLegend, cat. #806601). It was 6 times more CSF sample volume applied, indicating that the amount of structures at approximately 63 kDa in the nasal secretion sample is at least comparable to CSF or higher than CSF.

FIG. 13: pTau: directly comparing the recognized pTau structures in a nasal secretion sample and a CSF sample from the same individual reveals nearly identical structures at approximately 60 kDa, approximately 100 kDa, approximately 146 kDa, and approximately 230 kDa using the pTau specific antibody from Cell Signaling Technology (Phospho-Tau (Thr181) (D9F4G) Rabbit mAb, Cell Signaling, cat. #12885S). Six times more CSF sample volume than nasal secretion volume applied, indicating that the amount of structures at approximately 60 kDa, approximately 100 kDa, approximately 146 kDa and approximately 230 kDa is at least comparable to CSF or higher than CSF in the nasal secretion sample. Additional structures can be found in the nasal secretion sample at approximately 20 kDa.

FIG. 14: Analysis within two matrices (nasal fluid sample (FIG. 14A) and CSF (FIG. 14B)) of corresponding individuals show similar significant inter-protein correlations between Aβ42 to Aβ40 (r=0.63; p<0.001, N=79).

FIG. 15: Analysis within two matrices (nasal fluid sample (FIG. 15A) and CSF (FIG. 15B)) of corresponding individuals show similar significant inter-protein correlations between pTau to tTau (r=0.78, p<0.001, N=78).

FIG. 16: Analysis within two matrices (nasal fluid sample (FIG. 16A) and CSF (FIG. 16B)) of corresponding individuals show similar significant inter-protein correlations between Aβ42/Aβ40 to pTau (r=−0.345, p=0.003, N=73).

FIG. 17: Comparison of marker protein correlations in corresponding nasal fluid samples and CSF.

### DETAILED DESCRIPTION OF THE INVENTION

In the following the invention is described in more detail.

In particular, the invention relates to the following items:
1. A nasal fluid sample obtained from a subject, wherein the nasal fluid sample comprises the marker proteins β amyloid (Aβ), phosphorylated Tau (pTau) and/or total Tau (tTau).
2. The nasal fluid sample of item 1, wherein the nasal fluid sample is a purified nasal fluid sample.
3. The nasal fluid sample of item 1 or 2, wherein the nasal fluid sample of patients with cognitive impairment due to a neurodegenerative disease is characterized by a Aβ42 protein concentration that is decreased relative to the Aβ40 protein concentration.
4. The nasal fluid sample of any one of items 1 to 3, wherein the concentration of the marker protein(s) is (are) equivalent or at least 1.1-fold increased compared to a cerebrospinal fluid (CSF) sample obtained from the same subject.
5. The nasal fluid sample of any one of items 1 to 4, wherein the nasal fluid sample is characterized by a pTau protein concentration that is smaller than the tTau protein concentration.
6. The nasal fluid sample of any one of items 1 to 5, wherein pTau correlates positively with tTau and/or Aβ42 correlates positively with Aβ40.
7. The nasal fluid sample of any one of items 1 to 6, wherein the relative amount of Aβ42/Aβ40 is greater than 0 and less than 1.
8. The nasal fluid sample of any one of items 1 to 7, wherein the relative amount of Aβ42/Aβ40 correlates negatively with pTau and/or tTau.
9. The nasal fluid sample of any one of items 1 to 8, wherein the Aβ marker is characterised by a molecular weight of about 4 kDa, about 8 kDa, about 12 kDa, about 16 kDa, about 19 kDa, about 24 kDa, about 32 kDa, about 40 kDa, about 44 kDa, about 48 kDa, about 52 kDa, about 56 kDa, about 60 to 72 kDa, about 84 to 120 kDa and/or more than about 140 kDa.
10. The nasal fluid sample of any one of items 1 to 8 wherein the pTau marker is characterised by a molecular weight of about 20 kDa, about 30 kDa, about 38 kDa, about 55 to 62 kDa, about 96 to 106 kDa, and about 140 to 160 kDa and/or more than about 180 kDa.
11. The nasal fluid sample of any one of items 1 to 8 wherein tTau marker is characterised by a molecular weight of about 30 kDa, about 38 kDa, about 48 kDa, about 55 to 62 kDa, about 96 to 106 kDa, about 140 to 160 kDa and more than 160 kDa.
12. Use of the nasal fluid sample of any one of items 1 to 11 for the aid in diagnosis of a neurodegenerative disease.
13. A method for the aid in diagnosis of a neurodegenerative disease of a subject in a nasal fluid sample, wherein the method comprises the steps of:
   (a) determining the protein concentration of the marker proteins phosphorylated Tau (pTau), total Tau (tTau) and/or, optionally β amyloid (Aβ), in the nasal fluid sample as defined in any one of items 1 to 11;
   (b) comparing the protein concentrations of the marker proteins as determined in the nasal fluid sample with the protein concentration in controls; and
   (c) determining whether the values of the marker proteins are pathologically altered in relation to the protein concentration of controls.
14. The method of item 13, wherein the neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease.
15. The method of item 13 or 14, wherein the Aβ42 protein concentration that is decreased relative to the Aβ40 protein concentration is evidence of AD in a subject with cognitive impairment.
16. The method of any one of items 13 to 15, wherein the relative amount of Aβ42/Aβ40 which correlates negatively with pTau and/or tTau is evidence of a neurodegenerative disease in a subject.
17. The method of any one of items 13 to 15, wherein the relative amount of Aβ42/Aβ40 which correlates positively with pTau and/or tTau is evidence of a neurodegenerative disease in a subject.
18. The method of item 16, wherein any one of (i) or (ii) applies:
   (i) pTau correlates positively with tTau and Aβ42 correlates positively with Aβ40; and
   (ii) the relative amount of Aβ42/Aβ40 is greater than 0 and less than 1.
19. The method of item 17, wherein any one of (i) or (ii) applies:
   (i) pTau correlates positively with Aβ42/Aβ40 and Aβ42 correlates negatively with Aβ40; and
   (ii) the relative amount of Aβ42/Aβ40 is greater than 0 and less than 1.
20. The method of any one of items 13, 14, 16, 17, 18 or 19 wherein the neurodegenerative disease is AD.
21. The use of the nasal fluid sample of item 12, wherein the neurodegenerative disease is selected from the group consisting of Alzheimer's disease (AD), Parkinson's disease, chronic traumatic encephalopathy (CTE), frontotemporal dementia (FTD), Pick's disease and Creutzfeldt-Jakob's disease (CJD).

As mentioned above, the present invention provides a nasal fluid sample obtained from a subject comprising the marker protein(s) β amyloid (Abeta, also described as Aβ herein), phosphorylated Tau (pTau or also described as p-Tau) and/or total Tau (tTau or also described as t-Tau). The skilled person is aware that β amyloid (particularly Aβ42 and/or Aβ40), pTau (particularly pTau181, pTau231 and/or pTau217) and/or tTau are marker proteins reported to correlate with the diagnosis of neurodegenerative diseases. The term “comprising” as used herein is understood as to indicate that the nasal fluid sample comprises Aβ, pTau and/or tTau but may further comprise additional marker protein(s) associated with neurodegenerative diseases such as alpha synuclein, Neurofilament light (NFL), glial fibrillary acidic protein (GFAP), MTBR-Tau243, Triggering receptor expressed on myeloid cells 2 (sTREM2), Ubiquitin (Ub), Protein s100a, Apolipoprotein E ε4 (ApoE4), Superoxide dismutase 1 (SOD1), RNA-binding protein FUS/TLS (FUS), TAR DNA-binding protein 43 (TDP-43), Granulin (GRN), misfolded prion protein (PrPSc), mutant and wild type Huntington protein (Htt) and IgM and IgG against Epstein Barr Virus. Preferably, the additional marker protein(s) is (are) alpha synuclein, Neurofilament light (NFL), glial fibrillary acidic protein (GFAP), Protein s100a and/or Apolipoprotein E ε4 (ApoE4).

The terms “subject”, “patient” and “individual” may be used synonymously herein and refer to an organism (preferably a mammal, most preferably a human) that is diagnosed by using a nasal fluid sample according to the invention which is from said organism. Accordingly, the skilled person is well aware that the nasal fluid sample refers to an in vitro sample obtained from the subject.

The invention further relates to the nasal fluid sample comprising marker proteins that are characterized in a neurodegenerative disease, and the use of such a nasal fluid sample for the aid in the diagnosis of a neurodegenerative disease. The nasal fluid sample as described herein may be for use in a method of diagnosis or the aid in diagnosis of (a) neurodegenerative disease(s) in a subject. Accordingly, it is considered that the invention covers a nasal fluid sample, the use of the nasal fluid sample and a method for the aid in diagnosing neurodegenerative diseases by using the nasal fluid sample as described herein. Further, the use of the nasal fluid sample relates to i) selecting an appropriate therapy for a subject, ii) monitoring a subject's response to a treatment, iii) evaluating the likelihood of a positive response to a treatment, and/or iv) evaluating the likelihood of adverse events in response to a treatment.

The invention further relates to the use of the nasal fluid sample as described herein. Specifically, the invention relates to the use of the nasal fluid sample for the aid in diagnosis of a neurodegenerative disease. In accordance with the definitions described above, the use of the purified nasal fluid sample preferably relates to the aid in diagnosis of a neurodegenerative disease.

It is envisaged that what is said herein regarding the nasal fluid sample equally applies to the uses of the nasal fluid sample described herein, and to the methods for aid in diagnosis described herein.

The term “diagnosis” is used herein in the broadest sense and refers to the determination or the confirmation of a disease or condition of a subject, in particular from the results of various diagnostic procedures, including e.g., detecting protein levels or concentrations according to at least some embodiments of the invention in a biological sample obtained from the subject. The term “aid in diagnosis” is used herein as any method that can be used sequentially and/or simultaneously with a diagnosing method known in the art to pre-screen, supplement or validate the diagnosis of the disease or condition of the subject.

In the context of the present invention, the term “diagnosis” is to be understood as the clinical diagnosis. In the context of the present invention, the term “evidence of a neurodegenerative disease” is to be understood as the analyzed result based on the marker proteins in the nasal fluid sample obtained from a subject for the aid in a diagnosis of a neurodegenerative disease.

The skilled person is well-aware which diseases fall under the term “neurodegenerative diseases”. Examples of neurodegenerative diseases may be Alzheimer's disease (AD), Parkinson's disease (PD), chronic traumatic encephalopathy (CTE), frontotemporal dementia (FTD), Pick's disease, Creutzfeldt Jakob's disease (CJD), dementia with Lewy bodies, vascular dementia (VD), Huntington's Disease (HD), REM sleep behaviour disorder, multiple system atrophy (MSA), amyotrophic lateral sclerosis (ALS) and multiple sclerosis (MS). Preferably, a neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease (CJD). More preferably, a neurodegenerative disease is selected from the group consisting of Alzheimer's disease (AD), Parkinson's disease (PD) and chronic traumatic encephalopathy (CTE). Even more preferably, a neurodegenerative disease is selected from the group consisting of Alzheimer's disease (AD) and Parkinson's disease (PD). Most preferably, a neurodegenerative disease is Alzheimer's Disease (AD).

The invention provides a nasal fluid sample subject comprising the marker proteins β amyloid (Aβ), phosphorylated Tau (pTau) and/or total Tau (tTau) for use in a method of diagnosis of neurodegenerative diseases and the use of said nasal fluid sample for the diagnosis of a neurodegenerative disease. Further, the present invention relates to a nasal fluid sample subject comprising the marker proteins β amyloid (Aβ), phosphorylated Tau (pTau) and/or total Tau (tTau) for use in a method of diagnosis of neurodegenerative diseases and the use of said nasal fluid sample for the diagnosis of a neurodegenerative disease, wherein the neurodegenerative disease is preferably selected from the group consisting of Alzheimer's disease (AD), Parkinson's disease, chronic traumatic encephalopathy (CTE), frontotemporal dementia (FTD), Pick's disease and Creutzfeldt-Jakob's disease (CJD).

The subject may be treated according to the evidence of a neurodegenerative disease and/or may be selected e.g., for a study according to the evidence of a neurodegenerative disease.

The research framework for AD diagnosis, developed under the auspices of the National Institute on Aging and Alzheimer's Association (Jack (2018), Alzheimers. Dement., 14(4), 535-562), proposes to categorize individuals based on marker protein evidence of pathology, using the so-called A/T/N (amyloid, tau, neurodegeneration) classification system. According to the ATN system, each individual is rated for the presence of (i) Aβ42 (CSF Aβ42 or amyloid positron emission tomography-computed tomography (PET-CT): “A”), (ii) hyperphosphorylated Tau (CSF pTau181 or Tau PET-CT: “T”), and (iii) neurodegeneration (atrophy on structural magnetic resonance imaging (MRI), fluordeoxyglucose (FDG), PET-CT, or CSF total tau (tTau): “N”). This A/T/N classification is a globally accepted system to diagnose AD and initiate appropriate therapeutic consequences (Jack (2016), Neurology, 87(5), 539-547; Grontvedt (2020), J. Alzheimers Dis. 74, 829-837). According to the A/T/N classification the subject is classified in three binary categories A, T and N. That is, according to the A/T/N classification the subject is classified as A+ or A−, T+ or T− and N+ or N−. It is pointed out that for some applications it may be sufficient that the subject is only classified in one of the three categories. For example, the subject may be diagnosed with AD when the subject was classified only according to the A category and was classified as A+. In general, the subject suffers from AD when said subject is classified as A+.

The term “marker protein” as used herein refers to the proteins that may be associated with neurodegenerative diseases and are found in the nasal fluid sample and analyzed in the context of the herein described methods for the aid in diagnosis of a neurodegenerative disease of a subject.

The term “biomarker” as used herein refers to the proteins that may be associated with neurodegenerative diseases and are found in the nasal fluid sample and analyzed in the context of the herein described methods for the aid in diagnosis of a neurodegenerative disease of a subject.

The term “analyte” as used herein refers to the proteins that may be associated with neurodegenerative diseases and are found in the nasal fluid sample and analyzed in the context of the herein described methods for the aid in diagnosis of a neurodegenerative disease of a subject.

The term “marker protein” may be used interchangeably with “analyte”, “protein”, “marker” or “biomarker”. It is evident for the skilled person that depending on the context, “protein” may refer to a marker protein, an analyte or a biomarker that is analyzed in the herein described methods. However, “protein” can also refer to e.g., all proteins in a nasal fluid sample (i.e., all proteins in the nasal fluid sample including proteins that are not known to be associated with neurodegenerative diseases).

In the context of the present invention, the term “marker protein”, “analyte”, “marker” or “biomarker” may refer to the overall protein, e.g., β Amyloid, for which the protein concentration(s) of one or several marker protein(s) is (are) determined.

In other words, “marker protein”, “analyte” or “biomarker” may refer to the sum of several “marker proteins”, “analytes” or “biomarkers”. Accordingly, when it is herein referred to that a “marker protein”, “analyte” or a “biomarker” is analyzed/tested, this may mean that one or several marker protein(s) is (are) determined. For example, when it is herein referred to that the “marker protein”, “analyte” or “biomarker” β Amyloid is analyzed/tested, the protein concentrations of marker proteins Aβ40 and Aβ42 may be determined. But it may also refer to only one of the isoforms of β Amyloid, e.g., Aβ40 or Aβ42, for which the protein concentration is determined.

Accordingly, it is also evident for the skilled person that e.g., the term “(whole) protein level” may refer to e.g., the level of all proteins in a given sample. However, the term “(whole) protein level of β Amyloid” refers to the protein level of β Amyloid. The term “(whole) protein level of Aβ40” refers to the protein level of β Amyloid isoform Aβ40 and the term “(whole) protein level of Aβ42” refers to the protein level of β Amyloid isoform Aβ42. What is explained here for β Amyloid is of course also envisaged for pTau and tTau.

The terms “protein concentration”, “level”, “amount”, “protein level” and “protein amount” may be used synonymously herein. The terms “total protein concentration”, “total level”, “total amount”, “total protein level”, “total protein amount”, “whole protein concentration”, “whole protein level” and “whole protein amount” may be used synonymously herein. These terms may refer to a concentration or may be denoted as a concentration such as mg/mL or μM. However, they may also be denoted as arbitrary units. For example, it is envisaged herein that the analytes/marker proteins/biomarker/proteins of interest are measured via immunodetection. The analyte may be first contacted with a primary antibody specific for said analyte and after association of said primary antibody with said analyte a secondary antibody may be added specific for the primary antibody. The secondary antibody may be labeled with a moiety generating a detectable signal such as a fluorophore or a molecule generating chemiluminescence. The fluorescence or chemiluminescence may be detected and may be proportional to the amount of the analyte in the sample. The fluorescence or chemiluminescence may be denoted in arbitrary units.

It is envisaged that the analytes of interest in the sample can be separated e.g., via molecular weight prior to association with a primary antibody. In this case the detectable signal may be proportional to e.g., the different quaternary structures of the analyte in the sample.

It is also envisaged that the herein described methods comprise an electrophoretic technique. Accordingly, it is envisaged that the protein levels are determined via an electrophoretic technique. Accordingly, it is envisaged that the protein levels are determined via an electrophoretic technique comprising separation of the quaternary structures and/or isoforms.

The term “fold increase” is to be understood herein synonymously as “times”, where the numerical value is a positive number larger than 1. Specifically, value A having a 3 fold increase compared to value B, is to be understood as the value of A being equal to 3 times the value of B. Preferably, in the nasal fluid sample according to the present invention, the concentration of the marker protein(s) is (are) equivalent or at least 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6-fold increased compared to a cerebrospinal fluid (CSF) sample obtained from the same subject. More preferably, the concentration of the marker protein(s) is (are) at least 2-fold increased compared to a cerebrospinal fluid (CSF) sample obtained from the same subject. Even more preferably, the concentration of the marker protein(s) is (are) at least 4-fold increased compared to a cerebrospinal fluid (CSF) sample obtained from the same subject. Most preferably, the concentration of the marker protein(s) is (are) at least 6-fold increased compared to a cerebrospinal fluid (CSF) sample obtained from the same subject.

Accordingly, it is also evident for the skilled person that e.g., the term “(total) protein concentration” may refer to e.g., the concentration of all proteins in a given sample. However, the term “(total) protein concentration of β Amyloid” refers to the protein concentration of β Amyloid. The term “(total) protein concentration of Aβ40” refers to the protein concentration of β Amyloid isoform Aβ40 and the term “(total) protein concentration of Aβ42” refers to the protein concentration of β Amyloid isoform Aβ42. What is explained here for β Amyloid is of course also envisaged for pTau and tTau.

It is also envisaged that the terms “relative concentration”, “relative value”, “relative amount”, “relative level”, “relative protein amount” and “relative protein level” are synonymous in the context of the present invention.

The skilled person would appreciate that the concentrations of one or more marker proteins (or fragments thereof, or precursors, or fragments thereof) in the nasal fluid sample of a subject can be further analyzed to identify subjects that may be diagnosed with neurodegeneration. For instance, a ratio of one marker protein to another protein, i.e., a relative amount of one marker protein in respect of another marker protein, can be derived from the measured protein concentrations. Such ratio of marker proteins from a subject can be compared to the values of the corresponding marker proteins collected from a group of “control” subjects, herein also referred to as reference values, to determine whether the subject has any evidence of a neurodegenerative disease.

It is envisaged that the protein concentration of (a) marker protein(s) for key analytes is (are) determined in the herein described methods. In the context of the present invention, the term “protein concentration of a marker protein” means the protein concentration obtained from the herein described measurements for an analyte/marker protein/biomarker/protein and may include the total protein concentration of the analyte/marker protein/biomarker/protein.

In the context of the present invention the terms “marker protein for an analyte” and “marker protein of an analyte” herein may be used synonymously. Preferably, the key analytes are β Amyloid (e.g., β Amyloid 40 (Aβ40) and β Amyloid 42 (Aβ42) but also pan-Amyloid β) phosphorylated Tau (pTau), and total Tau (tTau). It is important to note that for each one of β Amyloid, pTau and tTau, more than one marker protein concentration may be determined in the context of the herein described methods. In the context of the present invention, additional marker proteins could be determined in the nasal fluid samples. In particular, the skilled person is aware of the specific marker protein(s) that are associated with the herein described neurodegenerative disease(s). For example, such additional marker protein(s) associated with neurodegenerative diseases may include alpha synuclein, Neurofilament light (NFL), glial fibrillary acidic protein (GFAP), MTBR-Tau243, Triggering receptor expressed on myeloid cells 2 (sTREM2), Ubiquitin (Ub), Protein s100a, Apolipoprotein E ε4 (ApoE4), Superoxide dismutase 1 (SOD1), RNA-binding protein FUS/TLS (FUS), TAR DNA-binding protein 43 (TDP-43), Granulin (GRN), misfolded prion protein (PrPSc), mutant and wild type Huntington protein (Htt) and IgM and IgG against Epstein Barr Virus. Preferably, the additional marker protein(s) is (are) alpha synuclein, Neurofilament light (NFL), glial fibrillary acidic protein (GFAP), Protein s100a and/or Apolipoprotein E ε4 (ApoE4).

As mentioned above, the nasal fluid sample described herein may comprise the marker protein Aβ (Aβ). A skilled person is aware that β Amyloid exists in the organism in different isoforms which can form different quaternary structures (oligomers). Isoforms of β amyloid may have any length of the β amyloid peptide 1 to 38, 1 to 39, 1 to 40, 1 to 41, 1 to 42, 1 to 43, or any N-terminally truncated isoforms x to 38, x to 39, x to 40, x to 41, x to 42, x to 43, wherein “x” is representative for any of the N-terminally truncated position, e.g. any position between amino acids positions 1 to 30, preferably 1 to 10, most preferably positions 2, 3 or 4. In the present invention, it is preferred that Aβ is present as the isoform β Amyloid 40 (Aβ40) and/or β Amyloid 42 (Aβ42).

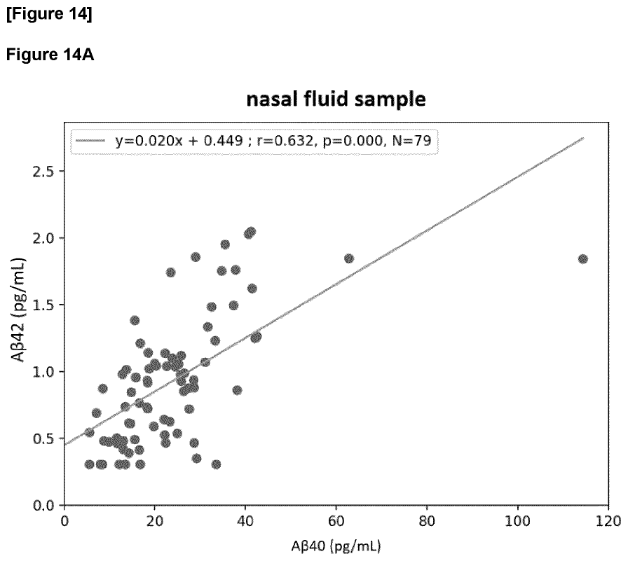

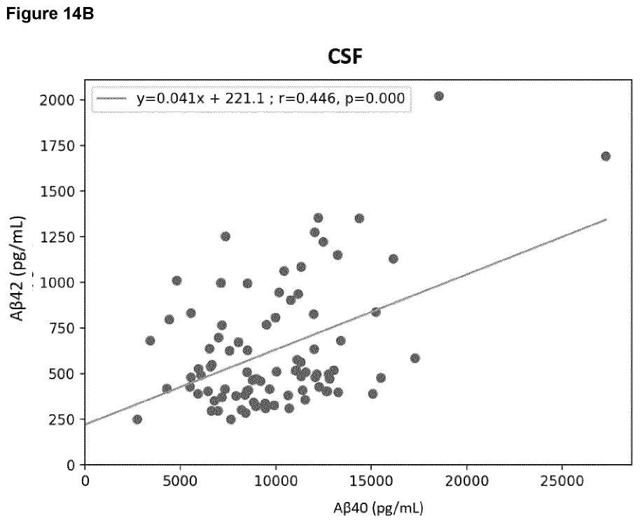

A skilled person is aware that Aβ40 and Aβ42 derive from a common precursor referred to as Amyloid-precursor protein (APP) and are produced in more or less identical quantities in the body. In the present invention, the nasal fluid sample as described herein may be characterized by an Aβ42 protein concentration that is increased relative to the Aβ40 protein concentration but is preferably characterized by an Aβ42 protein concentration that is decreased relative to the Aβ40 protein concentration, preferably in the case of neurodegeneration. The skilled person knows that neurodegenerative diseases are often associated with molecular-structural changes of Aβ42 which lead to the formation of plaques. The skilled person appreciates that in such a preferred embodiment, the Aβ42/Aβ40 protein ratio is decreased which is indicative for the presence of a neurodegenerative disease. Specifically, the Aβ42/Aβ40 protein ratio is decreased when compared to a healthy subject, i.e., a subject (preferably a mammal, most preferably a human) without any cognitive impairment due to a neurodegenerative disease. Further, the present invention relates to a purified nasal fluid sample obtained from a subject, wherein the nasal fluid sample of patients with cognitive impairment due to neurodegenerative disease is characterized by a Aβ42 protein concentration that is increased relative to the Aβ40 protein concentration or by a Aβ42 protein concentration that is decreased relative to the Aβ40 protein concentration. As shown in the appended Examples, particular FIG. 14A, a nasal fluid sample of patients with cognitive impairment due to neurodegenerative disease is characterized by a Aβ42 protein concentration that is decreased relative to the Aβ40 protein concentration.

As mentioned above, the nasal fluid sample described herein may comprise the marker protein pTau. A skilled person is aware that pTau exists in the organism in different isoforms. Isoforms of pTau are any phosphorylated version of Tau, including but not limited to pTau181, pTau202, pTau205, pTau217 pTau231, pTau199, pTau18, pTau396 and/or pTau422. Preferably, pTau is pTau181 and/or pTau231 and/or pTau217.

Isoforms of tTau include variants created by alternative splicing or proteolytic processing (fragments), this includes but is not limited to alternative splice forms 2N4R, 2N3R, 1N4R, 1N3R, 0N4R, 0N3R and/or fragments 1 to 314, 187 to 441, 1 to 255, 1 to 368, 151 to 421, 45 to 230, 243 to 441.

The present invention relates to the nasal fluid sample characterized by a pTau protein concentration that is smaller than the tTau protein concentration. The invention further provides a nasal fluid sample characterized by a pTau protein concentration that is smaller than the tTau protein concentration for use in a method in the aid of diagnosis of neurodegenerative diseases, and the use of said nasal fluid sample for the diagnosis of a neurodegenerative disease. The invention further relates to a method for the aid in diagnosis of a neurodegenerative disease in a subject by using the nasal fluid sample characterized by a pTau protein concentration that is smaller than the tTau protein concentration. The invention also relates to a method for the aid in diagnosis of a neurodegenerative disease in a subject by using the nasal fluid wherein the Aβ42 protein concentration that is increased or alternatively decreased relative to the Aβ40 protein concentration is evidence of AD in a subject with cognitive impairment. It is preferred that the Aβ42 protein concentration decreased relative to the Aβ40 protein concentration is evidence of AD in a subject with cognitive impairment.

The present invention relates to the nasal fluid sample characterized by a pTau protein concentration that is smaller than the tTau protein concentration, and wherein pTau correlates positively with tTau. The invention further provides a nasal fluid sample characterized by a pTau protein concentration that is smaller than the tTau protein concentration, wherein pTau correlates positively with tTau, for use in a method in the aid of diagnosis of neurodegenerative diseases, and the use of said nasal fluid sample for the diagnosis of a neurodegenerative disease. The invention further relates to a method for the aid in diagnosis of a neurodegenerative disease in a subject by using the nasal fluid sample, where in the relative amount of Aβ42/Aβ40 which correlates negatively with pTau and/or tTau is evidence of a neurodegenerative disease in a subject. Alternatively, the method for the aid in diagnosis of a neurodegenerative disease in a subject by using the nasal fluid sample may be characterized by a relative amount of Aβ42/Aβ40 which correlates positively with pTau and/or tTau is evidence of a neurodegenerative disease in a subject. The invention also relates to a method for the aid in diagnosis of a neurodegenerative disease in a subject by using the nasal fluid sample, wherein the relative amount of Aβ42/Aβ40 which correlates negatively with pTau and/or tTau is evidence of a neurodegenerative disease in a subject, and wherein any one of (i) or (ii) applies: (i) pTau correlates positively with tTau and Aβ42 correlates positively with Aβ40, alternatively pTau correlates positively with tTau and Aβ42 correlates negatively with Aβ40; and (ii) the relative amount of Aβ42/Aβ40 is greater than 0 and less than 1. Alternatively, the invention relates to a method for the aid in diagnosis of a neurodegenerative disease in a subject by using the nasal fluid sample, wherein the relative amount of Aβ42/Aβ40 which correlates positively with pTau and/or tTau is evidence of a neurodegenerative disease in a subject, and wherein any one of (i) or (ii) applies: (i) pTau correlates positively with Aβ42/Aβ40 and Aβ42 correlates negatively with Aβ40; and (ii) the relative amount of Aβ42/Aβ40 is greater than 0 and less than 1.

As shown in the appended examples, it is preferred that pTau correlates positively with tTau and Aβ42 correlates positively with Aβ40.

As explained herein, β Amyloid exists in the organism in different isoforms (e.g. Aβ40 and Aβ42). As also explained herein, these different isoforms in turn can form different quaternary structures (e.g. oligomers). Accordingly, there is a total concentration of a certain isoform (i.e., the sum of all quaternary structures of said isoform) and the concentration of a specific quaternary structure of said isoform. For example, for Aβ40, e.g., the quaternary structures with a molecular weight of 56 kDa and 97 kDa exist. Said quaternary structures are present in different concentrations (as evident from chemiluminescent signals in the Examples).

As an example, when it is explained herein that the protein concentration(s) of (a) marker protein(s) for β Amyloid is (are) determined, that means that the protein concentration(s) of one or several marker protein(s) is (are) determined. However, in addition, the protein concentration(s) for total β Amyloid may be determined. Thus, it is envisaged that the protein concentration of the marker protein for total β Amyloid, the protein concentration of the marker protein for Aβ40 and the protein concentration of the marker protein for Aβ42 is determined. All three protein concentrations of the marker proteins may then be used to determine the ratios of interest.

What is explained here for β Amyloid is of course also envisaged for pTau and tTau. Out of pTau, pTau-181 is preferred but other isoforms might also be preferred, e.g. pTau231 and pTau217.

The term “quaternary structure” is used herein in the broadest sense. A protein/polypeptide may have a primary structure, secondary structure, tertiary structure and quaternary structure. The quaternary structure of a protein is the association of several protein chains or polypeptide chains into a (closely packed) arrangement. For example, two proteins/polypeptides/oligopeptides may form a dimer and three proteins/polypeptides/oligopeptides may form a trimer. It is pointed out that quaternary structures as used herein refer to monomers and oligomers comprising two up to several thousand proteins/polypeptides. Quaternary structures may be distinguished by the number of protein chains or polypeptide chains comprised in the quaternary structure or by molecular weight (e.g. kDa) of the quaternary structure (e.g. as determined by running behavior in e.g. an electrophoretic technique). It is envisaged that the quaternary structures and/or isoforms may be separated between about 2 kDa and about 440 kDa. In other words, the technique comprising separation of the quaternary structures and/or isoforms may separate the quaternary structures and/or isoforms between a molecular weight of between about 2 kDa and about 440 kDa, such as about 2 to about 40 kDa, about 12 to about 230 kDa or about 66 to about 440 kDa, preferably about 2 to about 40 kDa, about 12 to about 230 kDa.

The protein concentration(s) of β Amyloid may comprise the whole protein level of total amyloid β and/or the protein level of quaternary structures of total amyloid β and/or the whole protein level of isoforms of amyloid β and/or the protein level of quaternary structures of isoforms of amyloid β. The protein concentration(s) of tTau may comprise the whole protein level of total tTau and/or the protein level of quaternary structures of total tTau and/or the whole protein level of isoforms of tTau and/or the protein level of quaternary structures of isoforms of tTau. The protein concentration(s) of pTau may comprise the whole protein level of total pTau and/or the protein level of quaternary structures of total pTau and/or the whole protein level of isoforms of pTau and/or the protein level of quaternary structures of isoforms of pTau.

As mentioned, the herein described methods may comprise that the protein levels are determined via a technique comprising separation of the quaternary structures and/or isoforms according to size, molecular weight or charge. For example, any form of gel electrophoresis, isoelectric focusing, size exclusion chromatography, or gel filtration chromatography.

Depending on the technique, device, buffer, individual/subject etc. the observed molecular weight in kDa may vary. It is envisaged that the observed molecular weight may vary up to 10%. Accordingly, the term “about” as used herein may mean that depicted value may vary +/−10%.

The molecular weight of a β amyloid may be about 4 kDa, about 19 kDa, about 24 kDa, about 32 kDa, about 40 kDa, about 44 kDa, about 48 kDa, about 52 kDa, about 56 kDa, about 60 to 72 kDa, about 84 to 120 kDa and/or more than about 140 kDa. Thus, it is envisaged that quaternary structures of β amyloid having the above-mentioned molecular weights are comprised in the protein concentration for β amyloid. Accordingly, the present invention relates to a nasal fluid sample wherein Aβ marker is characterised by a molecular weight of about 4 kDa, about 19 kDa, about 24 kDa, about 32 kDa, about 40 kDa, about 44 kDa, about 48 kDa, about 52 kDa, about 56 kDa, about 60 to 72 kDa, about 84 to 120 kDa and/or more than about 140 kDa.

The molecular weight of a pTau may be about 20 kDa, about 30 kDa, about 38 kDa, about 55 to 62 kDa, about 96 to 106 kDa, about 140 to 160 kDa and/or more than about 180 kDa. Thus, it is envisaged that quaternary structures of pTau having the above-mentioned molecular weights are comprised in the protein concentration for phosphorylated Tau. Accordingly, the present invention relates to a nasal fluid sample wherein the pTau marker is characterised by a molecular weight of about 30 kDa, about 38 kDa, about 55 to 62 kDa, about 96 to 106 kDa, and about 140 to 160 kDa.

The molecular weight of a tTau may be about 30 kDa, about 38 kDa, about 48 kDa, about 55 to 62 kDa, about 96 to 106 kDa, about 140 to 160 kDa and more than 160 kDa. Thus, it is envisaged that quaternary structures of tTau having the above-mentioned molecular weights are comprised in the protein concentration for tTau. Accordingly, the present invention relates to a nasal fluid sample wherein tTau marker is characterised by a molecular weight of tTau may have a molecular weight of about 30 kDa, about 38 kDa, about 48 kDa, about 55 to 62 kDa, about 96 to 106 kDa, about 140 to 160 kDa and more than 160 kDa.

The term “ratio” refers to the ratio of the protein amount or concentration between analytes, and is well known in the art. For example, the term “ratio of Aβ42/Aβ40” in the context of the current application is to be understood as the relative value of the Aβ42 protein concentration in respect of the Aβ40 protein concentration. The terms “ratio of Aβ42/Aβ40”, “Aβ42/Aβ40 ratio” and “relative amount of Aβ42/Aβ40” are used interchangeably herein.

The skilled person appreciates that preferably, the nasal fluid sample of the present invention is characterized by having a relative amount of Aβ42/Aβ40 that is greater than 0 and less than 1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1. More preferably, the nasal fluid sample of the present invention is characterized by having a relative amount of Aβ42/Aβ40 that is greater than 0.001 and less than 1. Even more preferably, the nasal fluid sample of the present invention is characterized by having a relative amount of Aβ42/Aβ40 that is greater than 0.003 and less than 0.75. Most preferably, the nasal fluid sample of the present invention is characterized by having a relative amount of Aβ42/Aβ40 that is greater than 0.005 and less than 0.75.

The relative amount/ratio of Aβ42/Aβ40 can be calculated by measuring the marker proteins Aβ40 and Aβ42 using an automated immunoassay platform such as the Simoa® Aβ42 Advantage kit, Simoa® Aβ40 Advantage kit (Quanterix, Billerica, MA, USA) and the Quanterix SIMOA SR-X Analyzer. The skilled person appreciates that when using a different automated immunoassay platform, the absolute values of the relative amount/ratio of Aβ42/Aβ40 may increase due to measurement system variations.

The term “control” in the context of the present invention is to be understood as a predefined level of the concentration of one or more marker proteins or a ratio derived from concentration of two or more marker proteins. The “control subject” may be a healthy subject or a subject without any cognitive impairment due to a neurodegenerative disease. Methods for obtaining the reference value from the group of “control subjects” selected are well known in the state of the art. The term “control” is interchangeable with the term “reference value”.

The skilled person appreciates that a subject without any cognitive impairment due to a neurodegenerative disease has a ratio of Aβ42/Aβ40 that is above a predefined cut off, in the sense, that the ratio is not decreased.

The term “correlation” is to be understood herein as the mutual relationship or association between two variables. For example, the variables are seen to have a positive correlation when an increase in one variable is associated with a proportional increase in the other variable, or when a decrease in one variable is associated with a proportional decrease in the other variable. Accordingly, the variables are seen to have a negative correlation when an increase in one variable is associated with a proportional decrease in the other variable, or when a decrease in one variable is associated with a proportional increase in the other variable. For example, the skilled person appreciates that when pTau correlates positively with tTau, it means that when pTau increases or decreases, tTau also increases or decreases, respectively.

The present invention relates to a nasal fluid sample, wherein the relative amount/ratio of Aβ42/Aβ40 correlates negatively with pTau and/or tTau. Accordingly, the skilled person appreciates that, when the relative amount/ratio of Aβ42/Aβ40 correlates negatively with pTau and/or tTau, it means that a decrease in the relative amount/ratio of Aβ42/Aβ40 correlates with an increase in the concentration of pTau and/or means that a decrease in the relative amount/ratio of Aβ42/Aβ40 correlates with an increase in the concentration of tTau. The invention further provides a nasal fluid sample wherein the relative amount/ratio of Aβ42/Aβ40 correlates negatively with pTau and/or tTau for use in a method in the aid of diagnosis of neurodegenerative diseases, and the use of said nasal fluid sample for the diagnosis of a neurodegenerative disease. The invention further relates to a method for the aid in diagnosis of a neurodegenerative disease in a subject by using the nasal fluid sample wherein the relative amount/ratio of Aβ42/Aβ40 correlates negatively with pTau and/or tTau. As shown in the appended examples, it is preferred that the relative amount/ratio of Aβ42/Aβ40 correlates negatively with pTau and/or tTau.

Alternatively, the present invention relates to a nasal fluid sample, wherein the relative amount/ratio of Aβ42/Aβ40 correlates positively with pTau and/or tTau. Accordingly, the skilled person appreciates that, when the relative amount/ratio of Aβ42/Aβ40 correlates positively with pTau and/or tTau, it means that a decrease in the relative amount/ratio of Aβ42/Aβ40 correlates with a decrease in the concentration of pTau and/or means that an increase in the relative amount/ratio of Aβ42/Aβ40 correlates with an increase in the concentration of tTau. The invention further provides a nasal fluid sample wherein the relative amount/ratio of Aβ42/Aβ40 correlates positively with pTau and/or tTau for use in a method in the aid of diagnosis of neurodegenerative diseases, and the use of said nasal fluid sample for the diagnosis of a neurodegenerative disease. The invention further relates to a method for the aid in diagnosis of a neurodegenerative disease in a subject by using the nasal fluid sample wherein the relative amount/ratio of Aβ42/Aβ40 correlates positively with pTau and/or tTau.

It is to be understood that variables can be values such as protein concentrations of marker proteins and/or ratios, i.e., relative amount of one marker protein in respect of another marker protein.

Statistical analysis tools could be used to calculate whether a correlation between the different variables exist. One possible statistical analysis tool is the Pearson's correlation coefficient. Pearson's correlation coefficient is a measurement of association that is well known in the art. Specifically, Pearson's correlation coefficient is the most common way of measuring a linear relationship between two variables, where a change in one variable is associated with a proportional change in the other variable. Pearson's correlation coefficient is commonly represented by “r” or the Greek letter ρ (rho). The statistical significance of a Pearson's correlation coefficient is commonly represented as a p-value, represented by “p”. The definition of a p-value and the method of calculation is well known in the art. A positive correlation coefficient (>0) describes a positive correlation between the two variables. A negative correlation coefficient (<0) describes a negative correlation between the two variables. When the correlation coefficient is 0, it describes no correlation between the two variables. The terms “Pearson's correlation coefficient”, “correlation coefficient”, “coefficient” are used herein interchangeably.

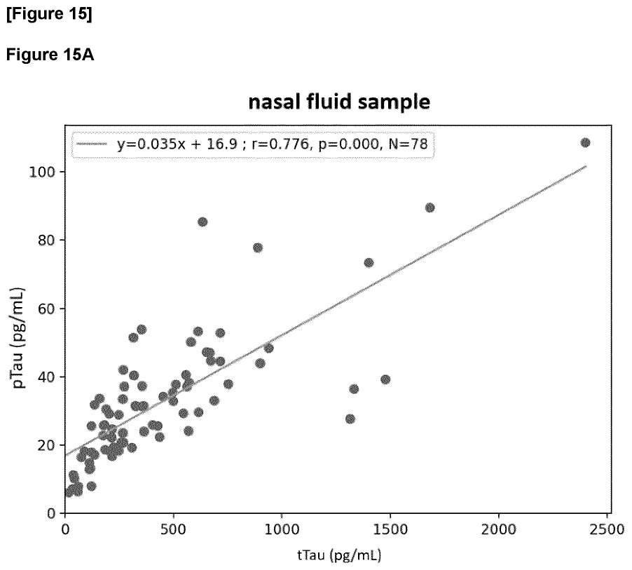

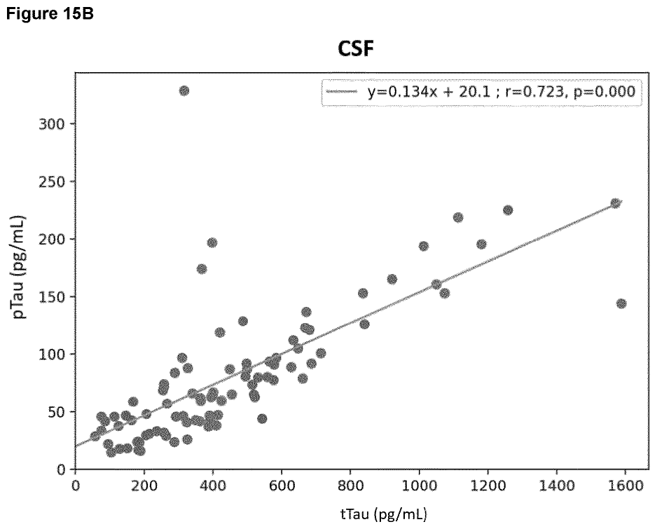

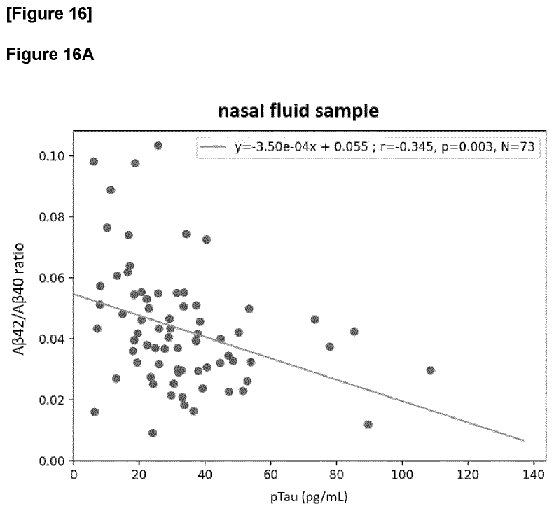

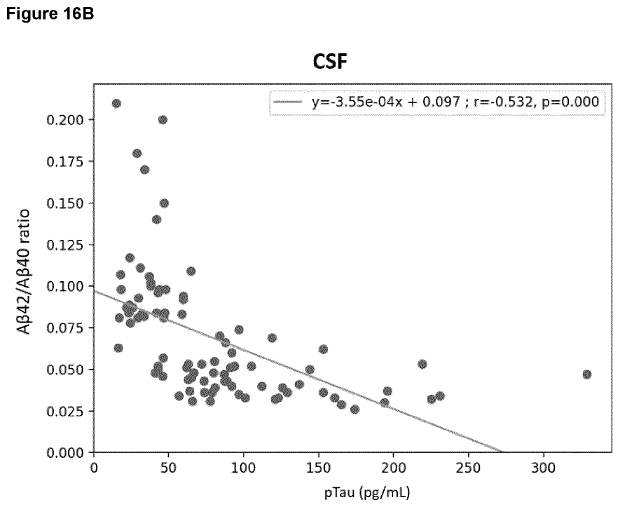

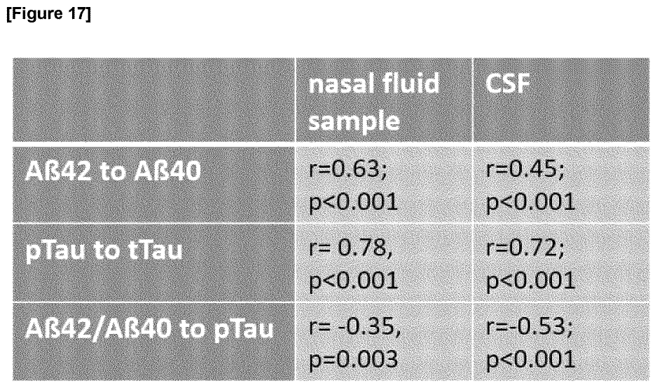

The skilled person appreciates, as disclosed in the Examples, that the correlation between the protein concentration or ratios of marker proteins from the nasal fluid sample according to the present invention can be measured as Pearson's correlation coefficients. Further, it is evident to the skilled person that the nasal fluid derived Pearson's correlation coefficients are comparable to those derived from CSF, allowing an equivalent diagnosis (FIGS. 14 to 17).

The term “pathologically altered” is used herein to mean values of the marker protein(s) that has (have) increased or decreased as characterized in CSF of subjects diagnosed with a neurodegenerative disease, in comparison to subjects without any cognitive impairment due to a neurodegenerative disease. For example, marker proteins measured from CSF of patients with AD have been reported to show lower Aβ42/Aβ40 protein concentration ratios, decrease in Aβ42 levels compared to Aβ40 levels, increase in pTau levels, and/or increase in tTau levels when compared to patients without AD.

The altered values of the marker proteins can be altered protein concentration of the marker proteins and/or changes in the relative amount of one or more marker protein(s) in relation to another marker protein.

The present invention relates to a method for the aid in diagnosis of a neurodegenerative disease of a subject in a nasal fluid sample, wherein the neurodegenerative disease is AD.

As described above, the present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls; and
(c) determining whether the values of the marker proteins are pathologically altered in relation to the protein concentration of controls.

Preferably, the present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls; and
(c) determining whether the values of the marker proteins are pathologically altered in relation to the protein concentration of controls,
wherein the neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease (CJD).

Further, the present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls; and
(c) determining whether the values of the marker proteins are pathologically altered in relation to the protein concentration of controls,
wherein the Aβ42 protein concentration that is increased or alternatively decreased relative to the Aβ40 protein concentration is evidence of AD in a subject with cognitive impairment.

The present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls; and
(c) determining whether the values of the marker proteins are pathologically altered in relation to the protein concentration of controls,
wherein the neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease (CJD).

It is also envisaged that the present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls; and
(c) determining whether the values of the marker proteins are pathologically altered in relation to the protein concentration of controls,
wherein any one of (i) or (ii) applies:
   (i) pTau correlates positively with tTau and Aβ42 correlates positively with Aβ40, alternatively pTau correlates positively with tTau and Aβ42 correlates negatively with Aβ40; and
   (ii) the relative amount of Aβ42/Aβ40 is greater than 0 and less than 1, and
wherein the neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease (CJD).

Alternatively, it is also envisaged that present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls; and
(c) determining whether the values of the marker proteins are pathologically altered in relation to the protein concentration of controls,
wherein any one of (i) or (ii) applies:
(i) pTau correlates positively with Aβ42/Aβ40 and Aβ42 correlates negatively with Aβ40; and
(ii) the relative amount of Aβ42/Aβ40 is greater than 0 and less than 1, and
wherein the neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease (CJD).

It is also envisaged that the present invention relates to a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined herein;
(b) comparing the protein concentrations of the marker proteins as determined in the nasal fluid sample with the protein concentration in controls;
(c) determining whether the values of the marker proteins are pathologically altered in relation to the protein concentration of controls;
(d) the Aβ42 protein concentration is increased or alternatively decreased relative to the Aβ40 protein concentration; and
(e) the relative amount of Aβ42/Aβ40 correlates negatively with pTau and/or tTau, alternatively wherein the relative amount of Aβ42/Aβ40 correlates positively with pTau and/or tTau
wherein any one of (i) or (ii) applies:
   (i) pTau correlates positively with tTau and Aβ42 correlates positively with Aβ40, alternatively pTau correlates positively with tTau and Aβ42 correlates negatively with Aβ40; and
   (ii) the relative amount of Aβ42/Aβ40 is greater than 0 and less than 1,
is evidence of AD in a subject with cognitive impairment.

The marker proteins/analytes are analyzed by techniques described herein (e.g. using electrophoretic techniques using different capillary sizes (low, mid, high molecular size)). Each measurement generates (a) signature(s) that is (are) specific to the respective protein and also specific for the individual from whom the sample was collected in the first place and also specific for the individual disease state. The signature(s) is (are) basically made up of a large number of values or parameters. These values or parameters include the total level of an analyte, a separation of the analyte by molecular weight into different quaternary structures (e.g. monomers and oligomers) and the level of the different quaternary structures (e.g. the level of monomers, the level of oligomers). The sum of the information results in (a) biomarker specific signature(s) (referred to in here as protein-specific biomarker signature) for each of the analytes, which includes the protein concentration of the analyte. The protein concentration/level for e.g. β amyloid is (are) also referred to herein as β amyloid concentration/level.

Measuring protein levels and determining protein concentrations in analytes can be determined by techniques known to the skilled in the art. Methods to measure protein levels and determine protein concentrations include, but are not limited to, Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of DJ-1 including but not limited to ligand binding, or interaction with other protein partners.

In one example, the protein concentration of (a) marker protein(s) in the nasal fluid sample obtained as described herein may be measured by BCA Assay (Pierce BCA Protein Assay Kit, Thermo Fisher Scientific, 10678484). One sample aliquot may be taken from the −80 °C freezer and equilibrated to room temperature (RT) on the bench for approximately 30 minutes. The protein concentration measuring assays should be performed according to the manufacturer's protocol and the concentration may be calculated in mg/mL. These results may further be used for the normalization of the total protein concentration in e.g., gel electrophoresis in following measurements.

To determine the protein levels of marker proteins, a corresponding sample may be run on an automated protein separation and immunodetection system (such as Simple Western™ Jess, BioTechne, 004-650), and optionally, on a traditional gel electrophoresis and Western Blot. An aliquot of a nasal fluid sample eluate may be thawed at room temperature for 30 minutes. An appropriate amount of volume may be taken from the sample so that the final total protein concentration in the assay may be about 0.25 mg/mL, about 0.5 mg/mL, about 0.75 mg/mL, about 1.0 mg/mL, about 1.25 mg/mL, about 1.5 mg/mL, about 1.75 mg/mL or about 2.0 mg/mL.

Preferably, the final total protein concentration in the assay is about 1.0 mg/mL. The sample may be diluted in sample buffer provided by the manufacturer such as BioTechne for the Simple Western™ Jess system. The diluted sample may be loaded into the Simple Western™ Jess system and may be started and run according to the manufacturer's instructions as single plex assay. Capillaries with different gel percentages suitable for low- (2 to 40 kDa Separation Module, 8×25 Capillary Cartridges, BioTechne, SM-W012), mid- (12 to 230 kDa Separation Module, 8×25 Capillary Cartridges, BioTechne, SM-W004), or high- (66 to 440 kDa Separation Module, 8×25 Capillary Cartridges, BioTechne, SM-W008) molecular weight separation may be used.

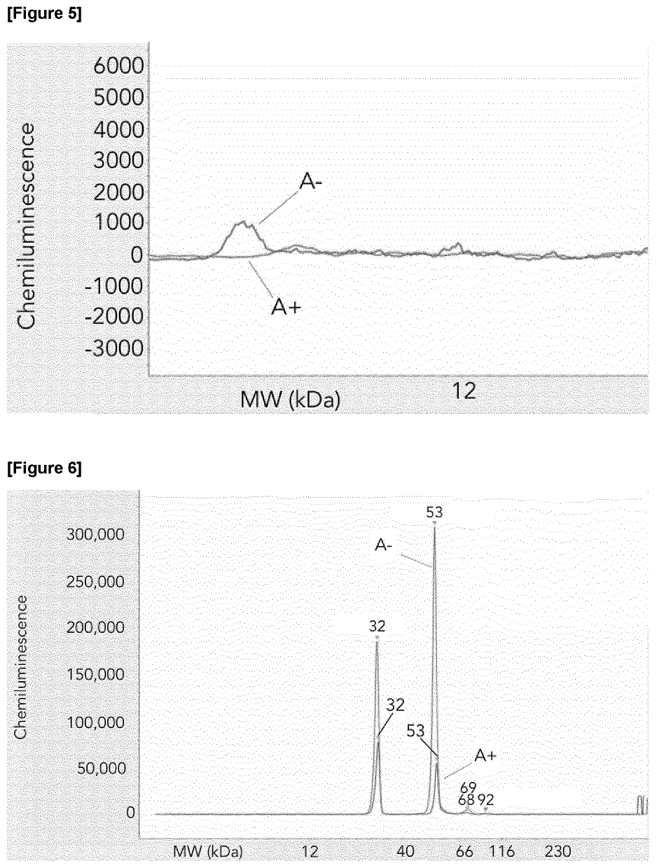

In the context of the present invention, the skilled person is aware of the means and methods for the detection of marker proteins such as Aβ, pTau and/or tTau in a sample. Exemplarily, for detection of Aβ40 the following primary antibodies may be used: Human Amyloid β (aa1-40) Antibody, BioTechne/R&D, MAB96181-100 and/or purified (azide-free) anti-β-Amyloid, 1-40 (11A50-B10), BioLegend, 805409. Both antibodies are specific to the C-terminus of Aβ40. An enzyme-linked secondary antibody (such as Anti-Mouse Detection Module, BioTechne, DM-003) may then be added followed by a chemiluminescent substrate. The Aβ40 protein amount may be measured, displaying information on the total level of the analyte and the level of different molecular weight (quaternary structures) of Aβ40 molecules within the same sample. This allows to simultaneously detect different oligomeric Aβ40 molecules and quantify them separately for each individual molecular weight, in total and/or in relation to each other. FIGS. 2 and 4 illustrate exemplary banding patterns of Aβ40 that may be measured in nasal fluid from patients with different clinical conditions. Each antibody-epitope recognition is represented by a chemiluminescent signal, thus higher signal intensities represent more antibody-epitope recognitions and thus represent higher levels of detected quaternary structures or overall levels of analyte. By calculating the area under the curve (AUC) the amount of antibody-epitope recognition may be measured and, thus, the level of a given quaternary structure or the total level of the protein can be back calculated. A graphical representation of the AUC, calculated from the chemiluminescent signal of the bound antibodies for Aβ40 may be generated. Oligomers of approximately 19 kDa, 32 kDa, 56 kDa and 100 kDa may be detected, as well as monomers at approximately 4 kDa. In FIG. 5, the signal of the Aβ40 4 kDa monomer band may be higher in patients without AD (classified as A−) in comparison to patients with clear AD (classified as A+). Without necessarily being bound by scientific theory it is believed that insoluble Aβ42 aggregates in the Alzheimer's patients sequester Aβ40 molecules leading to a decrease of detected Aβ40. The detected healthy monomeric species may be higher in patients without AD (A−) relative to patients with AD (A+).

FIG. 10 shows detected signals of Aβ40 oligomers of approximately 20 kDa, 32 kDa, 62 kDa, 98 kDa and 231 kDa from the nasal fluid sample. Detected signals of Aβ40 oligomers from corresponding CSF of the same individual were approximately 62 kDa, 87 kDa and 233 kDa. Aβ40 oligomers of approximately 32 kDa and 20 kDa were not detectable from the corresponding CSF. Comparing the quaternary/oligomeric structures of the same individual in corresponding samples nasal fluid and CSF one can observe the exact same distribution of oligomeric species at about 62 kDa and 231 kDa (233 kDa+/−10% technical deviation). The nasal fluid sample contains additional bands that cannot be seen in CSF.

For detection of Aβ42 the following primary antibodies may be used: Purified (azide-free) anti-β-Amyloid, 1-42 (12F4), BioLegend, 805501 and/or Anti Amyloid β42(43), Monoclonal Antibody (BC05), Fujifilm Wako, 010-26903. Both antibodies are specific to Aβ42 and may recognize one or several isoforms and oligomeric structures. An enzyme-linked secondary antibody (such as Anti-Mouse Detection Module, BioTechne, DM-003) may then be added followed by a chemiluminescent substrate. The protein amount may be measured, displaying information on the total level of the analyte and the level of different molecular weight of Aβ42 molecules (quaternary structures) within the same sample. This allows to simultaneously detect different oligomeric Aβ42 molecules and quantify them separately for each individual molecular weight, in total and/or in relation to each other. Comparing the different quaternary/oligomeric distributions and the relative protein amount of two different individuals/subjects, whereas one individual is suffering from a amyloid pathology (A+) and the other individual is not suffering from an amyloid-pathology (A−) (see FIG. 6), then one can observe that there may be an overall decrease in the detected total level of Aβ42 (calculated by the total area under the curve for the whole protein concentration) in an AD patient (classified as A+), relative to the total level of an individual without AD (A−). Also band ratios between the oligomers (53 kDa and 32 kDa in FIG. 6, may be altered.

Comparing the quaternary/oligomeric structures of the same individual in the two corresponding samples, i.e., nasal fluid and CSF (FIG. 11) from this individual, one can observe quaternary/oligomeric bands at approximately 99 kDa, 75 kDa, 56 kDa and 33 kDa in the nasal fluid sample, that were not determinable in the corresponding CSF sample of the same individual. There are much higher amounts of the respective oligomeric bands in nasal secretion than in the corresponding CSF.

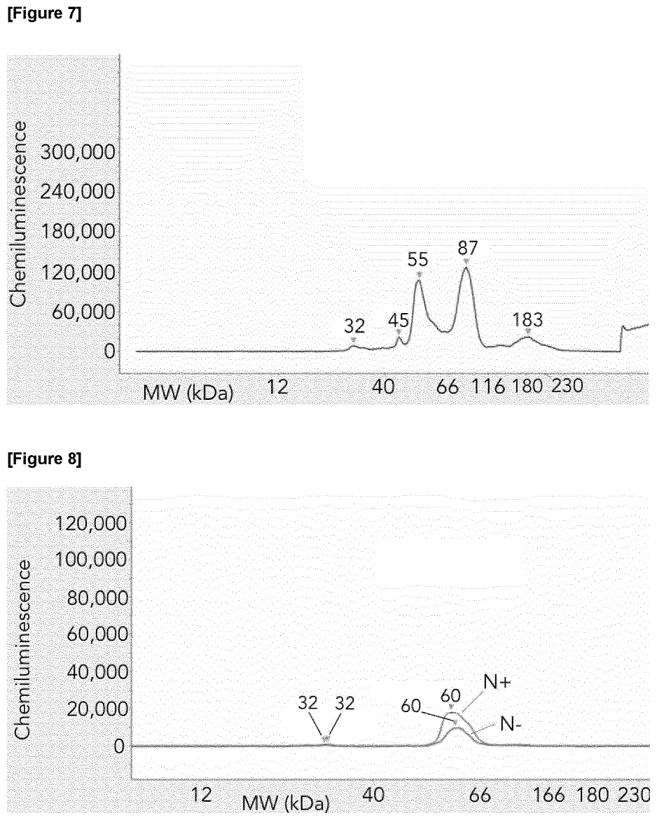

For detection of total Aβ the following primary antibodies may be used: primary antibody 6E10 (Purified anti-β-Amyloid, 1-16 Antibody (6E10), BioLegend, 803001) and/or monoclonal antibody to Amyloid β A4 (N-Terminus), Nanotools, 0315-100/bA4N-1E8 recognize all forms of Aβ. An enzyme-linked secondary antibody (such as Anti-Mouse Detection Module, BioTechne, DM-003) may then be added followed by a chemiluminescent substrate. The pan Aβ protein amount may be measured, displaying information on the total level of the analyte, as well as the level of different molecular weight of pan Aβ molecules (quaternary structures) within the same sample. This allows to simultaneously detect different oligomeric pan Aβ molecules and quantify them separately for each individual molecular weight, in total and in relation to each other (FIG. 7). The pan-Aβ antibody binds to an epitope that is more N-terminal than the species-specific antibodies described above. This allows for recognition of different quaternary structures compared to the antibodies specific for Aβ40 or Aβ42. It is envisaged that in A+ vs. A− individuals the ratio of the 45 kDa and 32 kDa bands, the total levels, and/or the levels of higher oligomeric structures such as the 183 kDa band are altered. It is further envisaged that these alterations can be used for classification e.g. A+ vs A−. The skilled person appreciates that in the context of the present invention, the terms “total Aβ”, “pan Aβ”, “total Aβ”, “pan Aβ”, “total β Amyloid”, “pan β Amyloid”, “total Amyloid β” and “pan Amyloid β” can be used synonymously.

For detection of tTau the following primary antibodies may be used: Purified anti-Tau, 404-441 Antibody (mouse), BioLegend, 806601 and/or Tau Monoclonal Antibody (HT7), Biotin (mouse), Thermo Fisher Scientific, MN1000B. Both are specific to tTau and recognize one or several isoforms and oligomeric structures. The enzyme-linked secondary antibody (Anti-Mouse Detection Module, BioTechne, DM-003) may then be added and followed by a chemiluminescent substrate. The tTau protein amount may be measured, displaying information on the total level of the analyte, as well as the level of quaternary structures or isoforms of tTau molecules within the same sample. This allows to simultaneously detect different oligomeric tTau molecules and quantify them separately for each individual molecular weight, in total and/or in relation to each other. Comparing the different oligomeric distributions and the relative protein amount of two different individuals/subjects, whereas one individual is suffering from neurodegenerative process (N+) and the other one is not suffering from a neurodegenerative process (N−) (see FIG. 8), one can observe that the detected level of canonical isoform of tTau, seen at approximately 60 kDa may be higher in patients with clear signs for neurodegeneration (classified as N+) than in patients without signs for neurodegeneration (classified as N−). Different other quaternary structures may be measured as well, e.g. total Tau dimers at 32 kDa. Comparing the quaternary/oligomeric structures of the same individual in the two corresponding samples, i.e., nasal fluid and CSF (FIG. 12) from this individual, one can observe a nearly identical oligomeric band at approximately 63 kDa in the nasal fluid sample and in the corresponding CSF sample. Given the fact that it was loaded 6-times more volume of CSF than corresponding volume from the nasal fluid, the levels of this quaternary/oligomeric band are at least equivalent to CSF or higher.

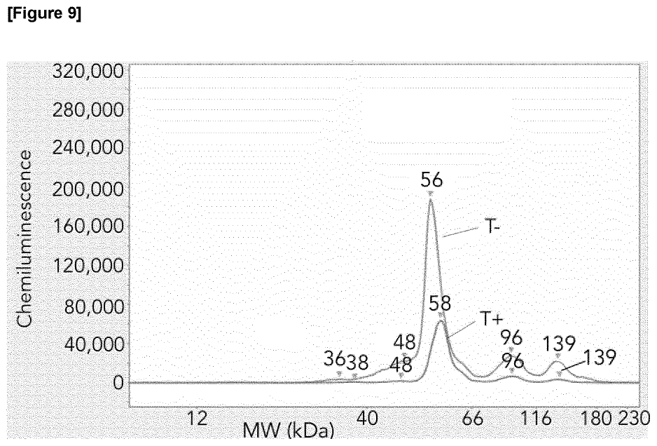

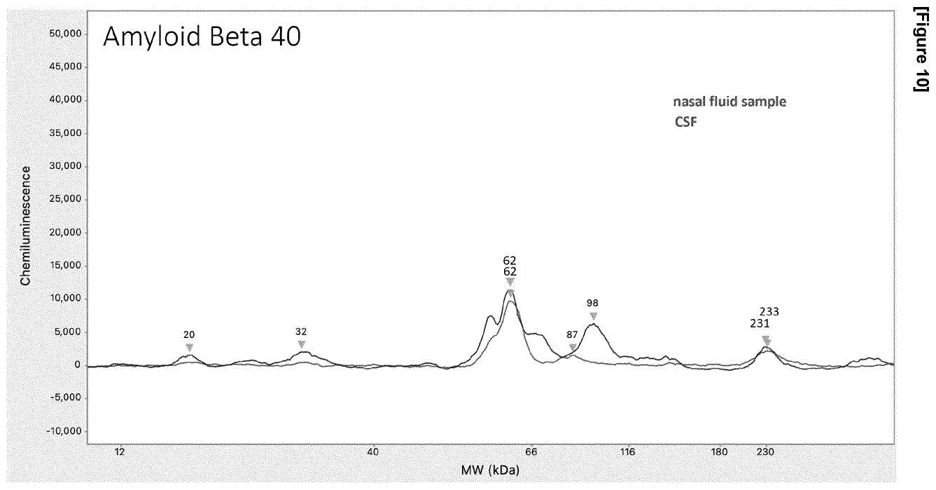

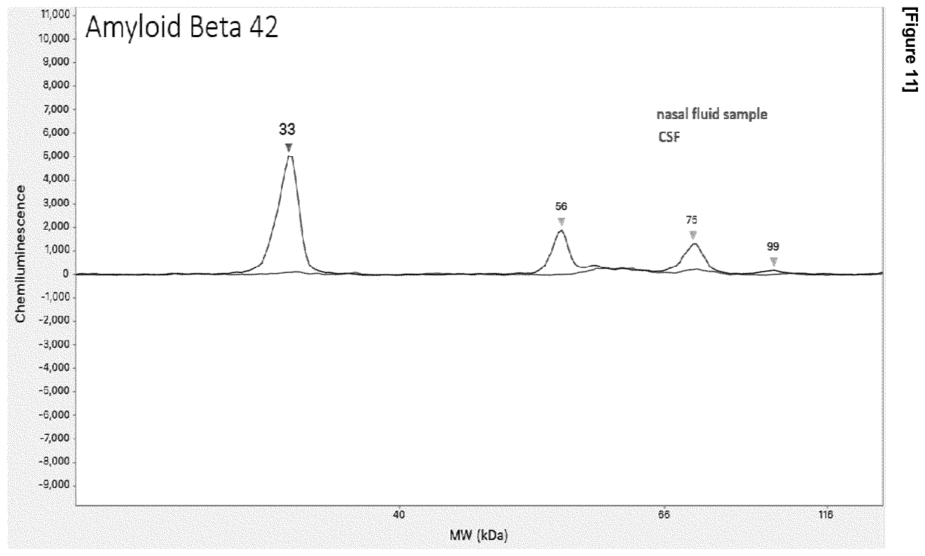

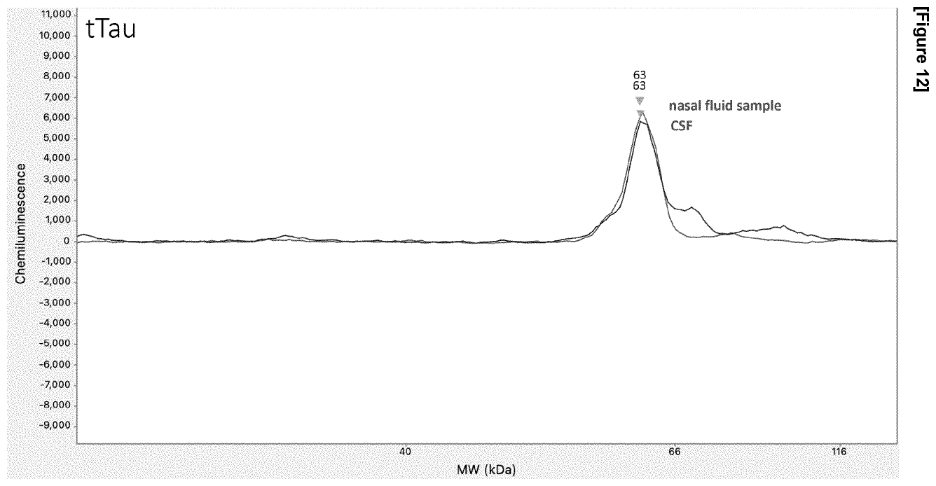

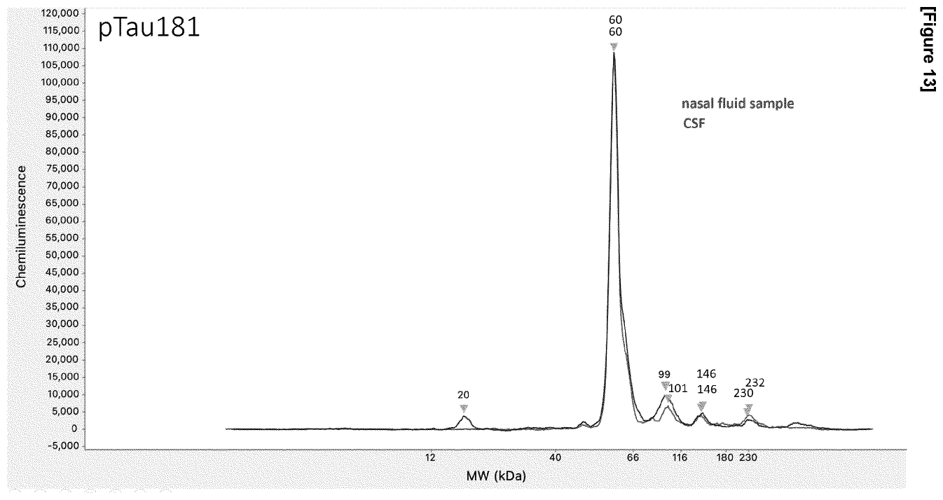

For detection of pTau the following primary antibody may be used: Phospho-Tau (Thr181) (D9F4G) Rabbit mAb, Cell Signaling, 12885S is specific to pTau-181 and recognizes one or several isoforms and oligomeric structures. An enzyme-linked secondary antibody (Anti-Rabbit Detection Module BioTechne, DM-001) may be added followed by a chemiluminescent substrate. The pTau protein amount may be measured, displaying information on the total level of the analyte and the level of quaternary structures with different molecular weight within the same sample. This allows to simultaneously detect different oligomeric pTau-181 molecules and quantify them separately for each individual molecular weight, in total and/or in relation to each other. Comparing the different oligomeric distributions and the relative protein amount of two different individuals/subjects, whereas one individual in this case is suffering from a Tau-pathology (T+) and the other one is not suffering from a Tau-pathology (T−) (see FIG. 9), one may observe that the total level of detected pTau may be decreased in this patient with clear Tau pathology (T+) compared to the other patient without Tau pathology (T−).

Comparing the quaternary/oligomeric structures of the same individual in the two corresponding samples, i.e., nasal fluid and CSF (FIG. 13), from this individual, one can observe nearly identical oligomeric bands at approximately 60 kDa, 100 kDa, 146 kDa and 230 kDa in the nasal fluid sample and in the corresponding CSF sample. Given the fact that it was loaded 6 times more volume of CSF than corresponding volume from the nasal fluid sample, the levels of these quaternary/oligomeric bands are at least equivalent to CSF or higher. An additional band at approximately 20 kDa may be seen in the nasal fluid sample that cannot be seen in the corresponding CSF sample, also indicating that the amount of this band is at least higher than in the corresponding CSF sample.

The present invention relates to a nasal fluid sample obtained from a subject, wherein the concentration of the marker protein is equivalent or at least 1.1-fold increased compared to a cerebrospinal (CSF) sample obtained from the same project. Specifically, the present invention relates to a purified nasal fluid sample obtained from a subject, wherein the concentration of the marker protein is equivalent or at least 1.1-fold increased compared to a cerebrospinal (CSF) sample obtained from the same project. Preferably, in the purified nasal fluid sample according to the present invention, the concentration of the marker protein(s) is (are) equivalent or at least 1.1-fold increased compared to a cerebrospinal fluid (CSF) sample obtained from the same subject. More preferably, in the purified nasal fluid sample according to the present invention, the concentration of the marker protein(s) is (are) at least 2-fold increased compared to a cerebrospinal fluid (CSF) sample obtained from the same subject. Even more preferably, in the purified nasal fluid sample according to the present invention, the concentration of the marker protein(s) is (are) at least 4-fold increased compared to a cerebrospinal fluid (CSF) sample obtained from the same subject. Most preferably, in the purified nasal fluid sample according to the present invention, the concentration of the marker protein(s) is (are) at least 6-fold increased compared to a cerebrospinal fluid (CSF) sample obtained from the same subject.

The skilled person in the art is aware that when analysing body fluids, matrix effect, or matrix interferences are often observed. Specifically, a matrix refers to the components of a sample other than the analyte of interest. The matrix can have a considerable effect on the way the analysis is conducted, and the quality of the results are obtained. Such effects are called matrix effects or matrix interferences.

A “purified nasal fluid sample” is to be understood herein as a nasal fluid sample in which the nasal fluid compounds causing above-described matrix effects and interferences to e.g., antibody epitope recognition, e.g., mucus proteins associated effects have been resolved prior to measuring the levels of one or more marker protein(s). Such nasal fluid compounds causing matrix effect—proteins may be, but not limited to, mucins, exosomes, lysozymes, lactotransferrin, immunoglobulins, albumin, cell debris, DNA fragments and bacteria. The removal of nasal fluid compounds causing matrix effects and interferences to obtain a purified nasal fluid sample can be done by techniques well known in the art. As an example, one step of purification can be done by using an electrophoretic system such as the Simple Western™ Jess system. A nasal fluid sample may be denatured and reduced by being boiled in appropriate buffer. Another step may be the use of a gel filter whilst applying an electric current to the gel and separated the molecules according to their sizes.

The skilled person is well aware that a nasal fluid sample does not mean the nasal fluid in the subject, but the nasal fluid in vitro or the nasal fluid sample obtained from a subject by the methods described herein. The skilled person is also well aware how the nasal fluid sample can be obtained from a subject. In the context of the present invention, a nasal fluid sample is synonymous as a nasal secretion eluate or nasal superficial lining fluid sample or nasal secretion sample.

In the context of the present invention, the skilled person understands that the terms “purified” and “refined”, or the terms “purify” and “refine” can be used interchangeably. Accordingly, “purification” and “refinement” also can be used synonymously herein.

Nasal fluid samples can be obtained by swapping, brushing, preferably by swapping or brushing the olfactory cleft, more preferably anywhere near the olfactory mucosa, nasal lavage/rinsing or biopsy of the olfactory mucosa. However, these techniques have certain disadvantages. Suction may result in loss of sample material, nasal lavage/rinsing may result in an uncontrollable loss of sample material into the nasopharynx and uncontrollable dilution and due to the fact that the actual area of interest, namely the vicinity of the olfactory cleft, only makes up a small part of the nose, washing out the whole nose is yielding in a further dilution of the proteins of interest. Brushing, swabbing and smearing is invasive, and the brush must be guided. Since the vicinity of the olfactory cleft is in sagittal alignment, without general anesthesia or local anesthesia one cannot reach the olfactory cleft manually, blood admixtures can alternate measurements, as there are organs that produce e.g. Aβ42 and plasma for example can contain Tau-proteins, therefore measurement can be falsified. Biopsy is invasive and since the area of interest is up to 23 cm\hat{ }2, a small biopsy of e.g. 1 mm\hat{ }2 is not displaying the whole pathology in its entirety. Accordingly, the collection of the nasal fluid sample by absorption is preferred. In context of the present invention, it is preferred that the nasal fluid sample is obtained from the vicinity of the olfactory cleft, in particular from the olfactory mucosa. The nasal fluid sample(s) may be obtained from one or both olfactory clefts, preferably both olfactory clefts.

No human has a completely symmetrical nose. Accordingly, the olfactory clefts and the olfactory mucosa in both nostrils may have a different size. Accordingly, it is preferred that the nasal fluid samples obtained from the vicinity of both olfactory clefts of a subject are pooled at some point before performing the analysis. Accordingly, the present invention relates to a nasal fluid obtained from a subject, wherein the nasal fluid sample comprises the marker protein(s) β amyloid (Aβ), phosphorylated Tau (pTau or p-Tau) and/or total Tau (tTau or t-Tau) obtained from the vicinity of the olfactory mucosa of the vicinity of both olfactory clefts of said subject.

It is preferred that an absorption material is placed in the vicinity of the olfactory cleft preferably on the vicinity of the olfactory mucosa and incubated for a certain amount of time in the vicinity of the olfactory cleft or on the vicinity of the olfactory mucosa. After incubation, the absorption material is recovered and the nasal fluid sample is isolated/obtained from the absorption material.

Accordingly, it is envisaged that in the herein described methods the nasal fluid sample is obtained by
a) placing an absorption material in the vicinity of the olfactory cleft(s);
b) incubating the absorption material in the vicinity of the olfactory cleft(s);
c) recovering the absorption material; and
d) isolating the nasal fluid sample from the absorption material.

It is also envisaged that the absorption material is placed in the vicinity of the olfactory cleft.

The absorption material used in the herein described methods is not particularly limited and the skilled person is readily capable of choosing suitable absorption material. Non limiting examples of absorption material used in context of the described methods may be synthetic material such as polyvinyl alcohol (PVA) or organic material such as cotton or a mixture.

It is preferred that the absorption material is in form of a sponge.

It is envisaged that the absorption material is incubated in the olfactory cleft for about 1 to 60 min, such as about 1 min, about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, about 40 min, about 41 min, about 42 min, about 43 min, about 44 min, about 45 min, about 46 min, about 47 min, about 48 min, about 49 min, about 50 min, about 51 min, about 52 min, about 53 min, about 54 min, about 55 min, about 56 min, about 57 min, about 58 min, about 59 min or about 60 min and all values in between such as about 14.7 min. It is preferred in context of the herein described methods that the absorption material is incubated in the olfactory cleft for 20 min.

Accordingly, it is envisaged that in the herein described methods the nasal fluid sample is obtained by
a) placing an absorption material in the vicinity of the olfactory cleft(s);
b) incubating the absorption material in the vicinity of the olfactory cleft(s) for about 1 to 60 min, preferably 20 min;
c) recovering the absorption material; and
d) isolating the nasal fluid samples from the absorption material.

It is envisaged that the nasal fluid sample is obtained using a the proprietarily developed absorbing material (AM) and a medical device to facilitate insertion of the AM at correct position (nosecollect®) as described in WO 2022/101311.

Accordingly, it is envisaged that in the herein described methods the nasal fluid sample is obtained by
a) placing an absorption material in the vicinity of the olfactory cleft(s);
b) incubating the absorption material in the olfactory cleft(s) for about 1 to 60 min, preferably 20 min;
c) recovering the absorption material; and
d) isolating the nasal fluid samples from the absorption material
e) purifying the nasal fluid samples from the nasal secretion eluate.

The operator collecting nasal secretion may be usually a psychiatrist or neurologist but may also be a different physician or trained medical personnel. It is preferred that the entire pathology is covered in both nostrils by collecting the sample from the vicinity of the whole olfactory mucosa area of about 23 cm\hat{ }2. It is preferred that the absorption material (e.g. in form of a sponge) is inserted in the sagittal orientation to cover the whole olfactory cleft in its entirety and that it expands in the caudal direction and not in coronal (laterally) when inserted in the vicinity of the olfactory cleft.

For collection of nasal fluid samples, the absorption material (e.g. polyvinyl alcohol (PVA)) may be either applied with nosecollect® (as described in e.g. WO 2022/101311) or manually into the vicinity of the olfactory cleft on both sides of a subject's nose by trained personnel. In the case of manual insertion, a Hartmann Nasal Speculum (13 cm, Karl Storz SE & Co. KG, 400500) as well as a Jansen Bayonet Nasal Forceps (16.5 cm, Karl Storz SE & Co. KG, 426516) may be used. A thread attached to the absorption material and hanging out of the nostril may be carefully fixed to the subject's cheek by an adhesive to prevent accidental displacement. The absorption material may be left in place for 20 to 30 minutes (or any other time mentioned herein, preferably 20 min). In case of dry mucosa, the subject may perform physical activity (walking around, climbing stairs), eating or drinking to stimulate nasal fluid secretion. Also other means might be suitable to increase nasal secretion production, e.g. the treatment of the nose with a saline spray previous to the sampling process.

In the following it is exemplarily disclosed how the nasal samples could be obtained and processed:

After incubation in the nose, the fixation of the threads may be loosened on a subject's cheek and the absorption material, which is saturated with nasal fluid, may be removed from both sides of the nose by pulling the threads. If the absorption material of one or both nostrils of a test person is completely bloody, it may be collected in separate tubes (Eppendorf 50 ml Protein LoBind Tube, Eppendorf, 0030122240) by the collecting personnel. If the absorption material of both sides is clean or only small blood spots are visible, the saturated absorption material of the second side (including the thread) may be combined with the absorption material from the first side into the same prelabeled tube (Eppendorf 50 ml Protein LoBind Tube, Eppendorf, 0030122240). The tubes with the removed absorption material may be collected upright in a suitable plastic box.

The box with the sample tubes may be held at room temperature (RT) for a maximum of 10 minutes. If several samples are taken and it is not guaranteed that no sample is at RT for more than 10 minutes, the box with the first samples may be immediately stored at −80 °C and new samples are successively added there. A temperature logger (Testo 184 T4, Testo SE & Co. KGaA, 05721844) may be added to the samples to guarantee compliance with the cold chain. The box, containing the collected nasal fluid samples may be stored at −80 °C with the lid facing up until shipment to the analytical laboratory. Storage in a −80 °C freezer is preferred, however other conditions like dry ice are also possible. In this case it should be assured that the complete box is surrounded by dry ice to guarantee equivalent temperature conditions all over the box. The box containing the collected samples and the temperature logger may be transferred into a styrofoam box with the lid facing up. The whole styrofoam box may be filled with dry ice. The sample box should be completely surrounded by the dry ice. Samples can then be shipped from e.g. the clinical site (overnight) to the analytical lab by e.g. a commercial logistics partner. The styrofoam box containing the nasal fluid samples that were collected as described herein may be opened at an analytical lab (preferably shortly after delivery). It may be checked if the collected samples are in good condition. This means, that they were frozen at −80 °C during the whole shipment (checked by the status of the remaining dry ice and the report of the included temperature logger) and the integrity of the tubes is fine (unbroken, sealed). The samples may then be stored at the analytical lab in specific racks or boxes in the −80 °C freezer until preprocessing. On the day of elution, the nasal secretion samples may be removed from the −80 °C freezer and thawed for 30 minutes at room temperature (RT). The thawed specimens may be visually inspected for signs of blood under a class II safety cabinet. Absorption material with blood spots may not be eluted together with bloodless absorption material. Therefore, blood spots may be cut out of the absorption material using a disinfected pair of scissors (microscopy scissors, curved, pointed/pointed, VWR, 233-1454) and a forceps (straight, blunt, VWR, 232-2116) before the absorption material from two sides of the nose of one subject are eluted together. The threads may be cut from the PVAs using a disinfected pair of scissors.

For elution, Pierce Centrifuge Columns (10 ml, Thermo Fisher Scientific, PIER89898) may be prepared by removing the silica membrane. The absorption material may be placed into these prepared columns, which may be put back into the original sample tube of the corresponding subject. If the blood spots cannot be removed, the absorption material of the two nostrils may be placed in separate centrifugation columns and tubes, respectively.

The tubes with the centrifugation column containing the absorption material may be centrifuged for 5 minutes at 4566 rcf at room temperature (RT). After that, the centrifuge column and the absorption material may be discarded. The complete volume of the eluted nasal fluid may be transferred from the original Eppendorf 50 ml Protein LoBind tube, that was used for centrifugation, into a pre-cooled Eppendorf 1.5 ml Protein LoBind tube (Eppendorf, 0030108116) and kept on ice.

The transferred eluates may be centrifuged for 10 minutes at 17000 rcf at RT in a suitable benchtop centrifuge to pellet the solid components. After centrifugation, the tubes may be placed on ice under a class II safety cabinet and the supernatant may be transferred into a new pre-cooled Eppendorf 1.5 ml Protein LoBind tube without pipetting solid or viscous portions. The volume of the transferred eluate may be estimated. If blood stains could not be removed from the absorption material before centrifugation, the absorption material of the two sides from the nose from one subject may be eluted separately. In this case, the two eluates from one subject continue to be processed separately. The eluate with the lowest value on the color scale may be analyzed alone or the eluate from both may be mixed for analysis.

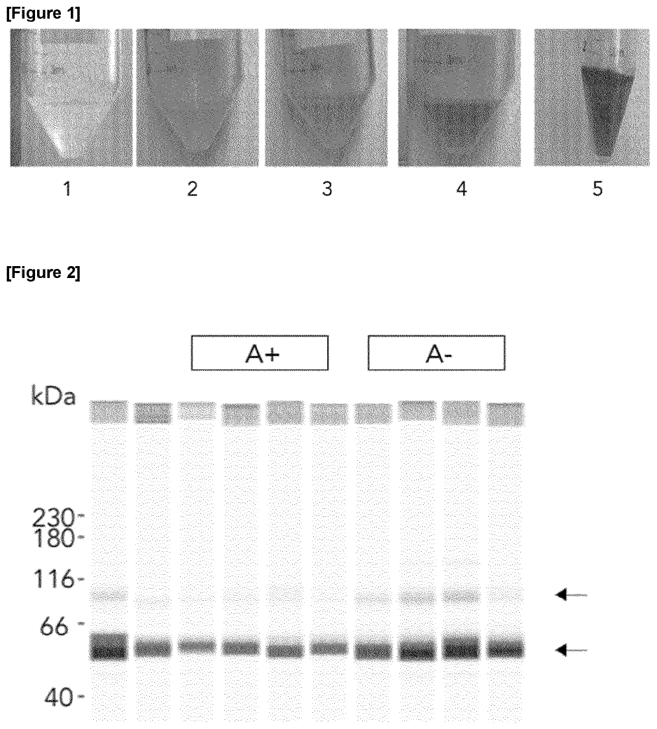

The eluates may be evaluated based on the available color scale (1-5) (FIG. 1). If a sample is rated color scale 5, the sample may not be further processed. Several working aliquots may be prepared in pre-cooled Eppendorf 1.5 ml Protein LoBind tubes under a class II safety cabinet. Samples and aliquots may be kept on ice during preparation. They may be stored at −80 °C for long term storage.

As mentioned above, the present invention also provides a method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample;
(b) comparing the protein concentrations of the marker proteins as determined in the nasal fluid sample with the protein concentration in controls; and
(c) determining whether the values of the marker proteins are pathologically altered in relation to the protein concentration of controls.

It is envisaged that in the herein described methods, the protein concentrations of marker proteins β amyloid, Aβ40 and/or Aβ42 are analyzed. In other words, it is envisaged that the isoforms of β amyloid are Aβ40 and/or Aβ42. It is also envisaged that in the herein described methods for the protein concentrations of marker proteins β amyloid, Aβ40, Aβ42 and/or total β amyloid are analyzed. Total β amyloid is also referred to pan β amyloid or pan Aβ. Total β amyloid may mean that all isoforms are analyzed, and the corresponding information is provided as the protein concentration for total β amyloid. This may be done with an antibody that does not distinguish between different isoform of β amyloid (e.g. as described in section 3.2.3 of the Examples) but detects all isoforms e.g. by binding to all isoforms. However, it is pointed out that for total β amyloid as used herein may also simply mean that, not a single isoform, but several isoforms that do not necessarily represent all β amyloid molecules in the sample are detected. For example, it is envisaged that e.g. an antibody detects β amyloid peptides 1 to 41, 1 to 42 (Aβ42) and 1 to 43 but not β amyloid peptides 1 to 38, 1 to 39 and 1 to 40 (Aβ40). Accordingly, the resulting protein concentration would comprise values regarding β amyloid peptides 1 to 41, 1 to 42 and 1 to 43. This may also be referred to as a total β amyloid protein concentration although it does not necessarily represent all β amyloid molecules in the sample.

Nasal fluid based measurements of core markers can be compared to CSF based measurements of the same core markers and be analyzed to find any evidence of a neurodegenerative disease such as AD. Nasal fluid samples and CSF samples may be collected from the same individuals, resulting in corresponding samples.

In the context of AD, these corresponding samples may,
i) demonstrate the quantities as well as inter-protein and intra-protein correlations (the pattern (signature) of the protein(s)) of the core AD marker protein(s);
ii) analyze the corresponding samples regarding the distribution and the quantities of their protein specific monomeric and oligomeric structure(s) of the AD marker protein(s) in the nasal fluid sample as well as in a corresponding CSF sample obtained from the same subject. Unexpectedly, it was found that the signature of the AD marker protein(s) Aβ40, tTau and pTau181 are nearly identical by a direct comparison. Notably, it was found that the signature of Aβ42 marker protein differs in the nasal fluid when compared to a corresponding CSF sample obtained from the same subject;
iii) distribution pattern of the protein specific monomeric- and oligomeric structures as well as their respective quantities within the two corresponding bodily fluids; and
iv) demonstrate neurodegenerative disease specific differences regarding relative protein amounts of the marker protein(s), and the distribution of monomeric- and oligomeric structures of the marker protein(s) within different subject with for example an underlying amyloid pathology compared to another subject without underlying amyloid pathology.

A suitable representative neurodegenerative disease to be assessed through the analysis of brain derived markers according to the context of the present invention could be AD for the following reasons:
i) it is the most common one and therefore a tremendous amount of data and knowledge exist;
ii) there are multiple markers whose quantities can up to date be reliably distinguished in CSF through in vitro diagnostic methods; and
iii) the AD specific inter-protein and intra-protein correlations in CSF, the protein pattern, has been evaluated in broad studies worldwide and the level of confidence for the trueness is high throughout the globe.

Pathological alterations of the marker proteins pTau and or tTau as well as pathological alterations of marker protein amyloid β can also be found in other neurodegenerative diseases. Tauopathies encompass a variety of different neurodegenerative disorders, which can be detected by different distinguished pattern, e.g. the pathological increase of tau-markers. Patients with PD as another example may show alterations of the marker proteins tau, amyloid β and alpha synuclein. Since the described proteins that can be found in the nasal fluid sample range from 4 kDa up to 400 kDa, all other CSF marker proteins within that range can be measured in the same nasal fluid sample as well. Thus, the claimed nasal fluid sample as well as their use in method for the aid of diagnosing neurodegenerative diseases will not be limited to the AD, but to the other known neurodegenerative diseases disclosed herein.

To measure the protein concentrations of marker proteins in CSF of the corresponding subjects, CSF may be collected at the clinical site by lumbar puncture, preferably in the morning and may be performed between the 3rd to 4th or 4th to 5th lumbar vertebrae. It may be collected in a polypropylene collection tube, discarding the first 20 drops, following international guidelines. Too much empty space in the tube should be avoided. Within the next 4 hours after collection, the CSF may be centrifuged for 10 minutes at approximately 2000 rcf, at room temperature (RT) and transferred into a new polypropylene collection tube. 1500 μl of the transferred CSF may be aliquoted into a separate polypropylene collection tube. CSF samples and aliquots may be stored in a suitable box at −80 °C within 4 hours after collection. The box containing the CSF aliquots and a temperature logger may be transferred into a styrofoam box with the lid facing up. The whole styrofoam box may be filled with dry ice. The sample box may be completely surrounded by the dry ice. Samples may then be shipped (overnight) to the analytical lab e.g. by a commercial logistics partner. The styrofoam box may be opened at the analytical lab shortly after delivery. It may be checked if the collected samples were in good condition. This means, that they should be frozen at −80 °C during the whole shipment (checked by the status of the remaining dry ice and the report of the included temperature logger) and the integrity of the tubes should be fine (unbroken, sealed). The samples may then be stored at the analytical lab in specific racks or boxes in the −80 °C freezer until preprocessing. The CSF aliquot may be thawed, and measurement may be performed using Immunoassay-based measurements, e.g. on a Fuji Rebio Lumipulse platform, according to the manufacturer's protocol for all core analytes: Amyloid-β40, Amyloid-β42, pTau181 and tTau.

As described herein nasal fluid samples may be subjected to techniques comprising separation of the quaternary structures and/or isoforms of the relevant marker proteins/analytes/biomarkers according to size, molecular weight or charge. It is envisaged that said techniques produce raw data that are processed further to measure protein concentration of the marker proteins/analytes/biomarkers.

Methods for separation of the quaternary structures and/or isoforms according to size, molecular weight or charge are well known to the skilled person in the art. As an example, the technique may be an electrophoretic technique such as ELISA, Simoa® Kits and/or a Quanterix SIMOA SR-X Analyzer (as exemplarily done in the Examples).

CSF based and nasal fluid based measurement results of all four analytes were used for subsequent data analysis.

Corresponding data sets were stored in Excel files and processed with Python scripts using standard libraries such as pandas and NumPy. Linear and logistic regression were calculated using the sklearn package. Graphics were generated using the matplotlib and seaborn packages. For quantitative analysis, primarily these methods were used to draw conclusions from the data. P-value for the probability of measuring the same or larger difference between the observed samples if the underlying distribution is indeed the same; smaller values indicate a significant difference. Values of p<0.05 are described as significant. It was also verified that non-parametric alternatives to Student's t (Mann-Whitney, Welch) lead to similar values and conclusions. When comparing two variables (e.g., different marker/analytes) from the same cohort, linear regression was used. The outcome (besides a fitted line giving the optimal linear relationship between the quantities) is Pearson's “r”, that is a parameter for the goodness of fit, and Pearson's p is a parameter for the statistical significance. The parameter “r” indicates to what degree one of the quantities is determined by the other, with a range of 0.1 to 0.3 counting as a weak correlation, 0.3 to 0.5 as moderate correlation, and 0.5 and more as strong correlation. The significance p again indicates the probability of measuring such a degree of correlation or more by chance if the quantities were independent.

The following describes measuring protein amount or concentration of marker proteins when using a technique comprising separation of the quaternary structures and/or isoforms of the relevant marker proteins/analytes according to molecular weight and immunodetection.

The corresponding nasal fluid and CSF samples were run on an automated protein separation and immunodetection system (Simple Western™ Jess, BioTechne, 004-650) and optionally on traditional gel electrophoresis and Western Blot to demonstrate the nearly identical distribution of protein specific monomeric- and oligomeric structures as well as to demonstrate the respective quantities within these different quaternary structures and the relations thereof. The protocols described refer to the automated system. The nasal fluid aliquots were initially thawed at room temperature for 30 minutes. An appropriate amount of volume was taken of each of the nasal fluid samples so that the final total protein concentration in the assay was 1.25 mg/ml. The sample was diluted in sample buffer provided by the manufacturer. The diluted sample was loaded into the Simple Western™ Jess system and the system was started and run according to the manufacturer's instructions as single plex assay. Capillaries with different gel percentages suitable for low- (2 to 40 kDa Separation Module, 8×25 Capillary Cartridges, BioTechne, SM-W012), mid- (12 to 230 kDa Separation Module, 8×25 Capillary Cartridges, BioTechne, SM-W004), or high- (66 to 440 kDa Separation Module, 8×25 Capillary Cartridges, BioTechne, SM-W008) molecular weight separation were used. Primary antibodies specific to the core AD marker proteins were used that recognizes one or several marker protein specific isoforms and oligomeric structures. An enzyme-linked secondary antibody is then added followed by a chemiluminescent substrate. The marker protein specific biomarker signature is measured, displaying information on i) the total level of the analyte and ii) the maximum position as well as the quantities of quaternary structures at different molecular weight within the same sample. As shown for example in FIGS. 10,11,12 and 13, this allows to simultaneously detect mono- and oligomeric molecules and quantify them separately for each individual molecular weight, in total and/or in relation to each other. On the same measurement platform corresponding CSF samples from the same individuals were applied according to the above-mentioned protocol with the only exception that 6 times the volume of CSF was applied compared to the applied volume of the nasal fluid samples. As previously described CSF was collected in the lumbar area. In CSF the core protein marker for AD gets diluted in a large volume, therefore the fluid itself is not very protein rich.

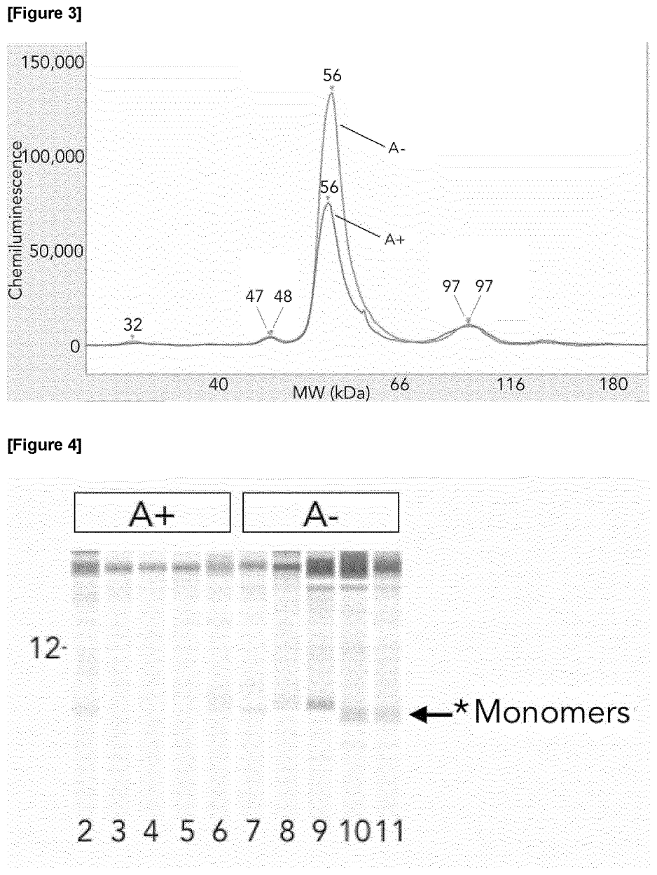

Each measurement of a corresponding body fluid sample set on the automated protein separation and immunodetection system yielded in a set of data for each marker. As shown in FIGS. 10,11,12 and 13, the measurements are displayed in a spectrum that gives an intensity (corresponding to the concentration of protein-antibody complexes, displayed on the y-axis) as a function of the molecular weight (monomeric or oligomeric structures displayed on the x-axis). A software can provide a way to apply a baseline correction algorithm to the spectra, and the corrected spectra may be used as displayed in FIGS. 3, 5, 6, 7, 8, 9, 10, 11, 12 and 13. By comparing the total intensity in the generated spectrum by numerically integrating the intensity over the molecular weight in the quantifiable range—i.e., calculate the area under the curve (AUC) with the correct weighting, one may also calculate the contribution to the signal from individual bands or groups of bands by applying a fit algorithm that fits a set of peaks.

The result for each sample and run is a set of measurements for one analyte, which is called the protein-specific biomarker signature which includes, but is not limited to, the total intensity and the intensity from each of the displayed bands.

So besides the intensities of each band, which is equal to the quantities of each molecular size, the distribution of these bands within a certain molecular range can be seen. Additionally this allows to evaluate ratios or relations of different band sizes and quantities towards each other.

Since similarities and or differences between the two body fluids can be seen by eye in FIGS. 10,11,12, and 13, no additional data are provided here to compare corresponding nasal fluid and CSF based measurements.

It is also envisaged herein that the marker protein/analyte measurements are calibrated for improved quantification of the protein-specific biomarker signature.

The “Run Processing SW” and “Sample Processing SW” may be used as command-line tools that process run data and generate sample reports on demand. They may also be incorporated in an automated setup, where new data from runs of automated measurement devices is processed as soon as it becomes available, and sample reports comprising the measurements of protein levels/concentrations as well as the position and total intensities of each different band or signature are generated as soon as the necessary measurements for that sample are complete.

As described herein, certain measured or calculated values of the protein-specific biomarker signature maybe used to determine the protein concentration(s) of one or more marker protein(s) and/or relative amount/ratio of one marker protein in relation to another marker protein. It is evident for the skilled person that the corresponding protein-specific biomarker signature comprises said values or corresponding information that allow calculation of said values.

It is evident for the skilled person that further physiological parameters and/or further markers of the neurodegenerative disease are measured in the herein described methods.

Thus, it is envisaged that the herein described methods may further comprise the measurement of further physiological parameters and/or further markers of the neurodegenerative disease.

Other physiological parameters and/or further markers of the neurodegenerative disease may be included to further streamline the diagnosis and support the physician's decision. Additional parameters could include, but are not limited to, age, cognitive performance measured by psychometrical means (e.g. Mini Mental Status Test (MMST), risk factors like genetic predisposition, sleeping disorders, Neurofilament light (NFL), glial fibrillary acidic protein (GFAP), alpha synuclein, MTBR-Tau243, Apolipoprotein E ε4 (ApoE4), protein S100b, protein S100a, neurogranin, Triggering receptor expressed on myeloid cells 2 (sTREM2),, Interleukin, Ubiquitin (Ub), Superoxide dismutase 1 (SOD1), RNA-binding protein FUS/TLS (FUS), TAR DNA-binding protein 43 (TDP-43), Granulin (GRN), misfolded prion protein (PrPSc), mutant and wild type Huntington protein (Htt) and IgM and IgG against Epstein Barr Virus, Immunoglobulins, imaging parameters like reduced brain volume, vascular pathologies, enlarged cerebrospinal fluid spaces or the general consumption of drugs like smoking and alcohol.

It is envisaged that the evidence identified based on the herein described methods can aid in diagnosis of a neurodegenerative disease. Accordingly, the herein described methods may further comprise pre-screening subjects for a diagnosis and/or prediction of a neurodegenerative disease. Further, the evidence of a neurodegenerative disease identified based on the nasal fluid sample and the herein described methods may be combined with a corresponding diagnosis to select an appropriate treatment for the neurodegenerative disease.

Reliable and approved nasal fluid-based identification of pathologically altered values of marker proteins for the aid in diagnosis of neurodegenerative diseases may become fully validated in the near future. The robustness of the neurodegeneration marker proteins detected in the nasal fluid described herein allows a subject to be effectively identified for a follow-up diagnosis or treatment and also monitored over time to evaluate therapeutic effects or side effects that may be suitable to evaluate therapy efficacy or therapy failure as well.

Treatments can include, but are not limited to, anti-amyloid-based antibody drug therapies like Lecanemab. Lecanemab (Leqembi®), for example, is approved in the United States to treat early AD (mild cognitive impairment [MCI] due to AD or mild AD dementia) with confirmed brain amyloid pathology. Treatment subjects should have amyloid pathology, either demonstrated by amyloid positron emission tomography (PET) results or cerebrospinal fluid (CSF) tests indicative of AD. Also the confirmation of efficacy of anti-amyloid drugs like Lecanemab in the clinical setting could be feasible since the change in the rate of decline is relatively subtle but a change in the values of the marker proteins could reliably be detected and thus, be a readout of efficacy of a selected treatment, also supporting dose adaptations and thus reduce side effects.

It is also envisaged that the herein described nasal fluid samples and methods can be used to monitor a therapeutic response. Accordingly, the values of the marker proteins of the herein described methods may be used to monitor a therapeutic response. For example, if a patient, from their nose fluid sample, is identified to have pathologically altered values of marker proteins, the patient may then be selected based on their characterization for e.g. a given anti-amyloid treatment such as Lecanemab. In a successful treatment, the patient would be expected to, for example, show one or more of the following changes: alterations in one or more of the oligomeric bands as well as alterations of the relative protein amounts of one or more of the marker proteins Aβ42, Aβ40, pTau and tTau, e.g., increased level of relative protein amount of Aβ42, resulting in changes in the Aβ42/Aβ40 ratio and possibly also decreased levels of relative amount of pTau and/or tTau as well as changes in the oligomeric banding pattern of these marker proteins.

Another possibility could be that a patient's profile does not change but, on the contrary, remains the same over time, which can also be seen as a therapeutic success and can indicate a steady state, i.e. the progression of the disease could be significantly delayed or even stopped.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

The present invention is further described by reference to the following non-limiting figures and examples.

The following Examples illustrate the invention

### 1. Collection, Storage, Transport, Elution and Preparation of Nasal Fluid Samples

Samples were collected at four different clinical sites. Therefore, absorption material (AM) polyvinyl alcohol (PVA) was either applied with nosecollect® or manually applied into the vicinity of the olfactory cleft on both sides of a subject's nose (N=2) by trained personnel. In the case of manual insertion, a Hartmann Nasal Speculum (13 cm, Karl Storz SE & Co. KG, 400500) as well as a Jansen Bayonet Nasal Forceps (16.5 cm, Karl Storz SE & Co. KG, 426516) were used. The thread attached to the AM and hanging out of the nostril was carefully fixed to the subject's cheek by an adhesive to prevent accidental displacement. The AM was left in place for 20 to 30 minutes. In case of dry mucosa, the subject performed physical activity (walking around, climbing stairs), eating or drinking to stimulate nasal secretion. After incubation in the nose, the fixation of the threads was loosened on subject's cheek and the AM, which was saturated with nasal secretion, was removed from both sides of the nose by pulling the threads. If one or both AMs of a test person were completely bloody, they were collected in separate tubes (Eppendorf 50 ml Protein LoBind Tube, Eppendorf, 0030122240) by the collecting personnel.
The tubes with the removed AM were collected upright in a suitable plastic box. The box with the sample tubes was held at room temperature (RT) for a maximum of 10 minutes and immediately stored at −80 °C. A temperature logger (Testo 184 T4, Testo SE & Co. KGaA, 05721844) was added to the samples to guarantee compliance with the cold chain. The box, containing the collected nasal fluid samples was stored at −80 °C with the lid facing up until shipment to the analytical laboratory. Storage in a −80 °C freezer was preferred, however other conditions like dry ice were also possible. In this case it was assured that the complete box was surrounded by dry ice to guarantee equivalent temperature conditions all over the box.
The box containing the collected samples and the temperature logger was transferred into a styrofoam box with the lid facing up. The whole styrofoam box was filled with dry ice. The sample box was completely surrounded by the dry ice. Samples were then shipped over night to the analytical lab by a commercial logistics partner.
The styrofoam box was opened at the analytical lab shortly after delivery. It was checked if the collected samples were in good condition. This means, that they were frozen at −80 °C during the whole shipment (checked by the status of the remaining dry ice and the report of the included temperature logger) and the integrity of the tubes was fine (unbroken, sealed). The samples were then stored at the analytical lab in specific racks or boxes in the −80 °C freezer until preprocessing.
On the day of elution, the nasal secretion samples were removed from the −80 °C freezer and thawed for 30 minutes at room temperature (RT). The thawed specimens were visually inspected for signs of blood under a class II safety cabinet. AM with blood spots was never eluted together with bloodless AM. Therefore, blood spots were cut out of the AM using a disinfected pair of scissors (microscopy scissors, curved, pointed/pointed, VWR, 233-1454) and a forceps (straight, blunt, VWR, 232-2116) before the two AMs from one subject were eluted together. The threads were cut from the PVAs using a disinfected pair of scissors. For elution, Pierce Centrifuge Columns (10 ml, Thermo Fisher Scientific, PIER89898) were prepared by removing the silica membrane. Both AMs were placed into these prepared columns, which were put back into the original sample tube of the corresponding subject. If the blood spots cannot be removed, the two AMs were placed in separate centrifugation columns and tubes, respectively. The tubes with the centrifugation column containing the AMs were centrifuged for 5 minutes at 4566 rcf at room temperature (RT). After that, the centrifuge column and the AMs were discarded. The complete volume of the eluted nasal secretion was transferred from the original Eppendorf 50 ml Protein LoBind tube, that was used for centrifugation, into a pre-cooled Eppendorf 1.5 ml Protein LoBind tube (Eppendorf, 0030108116) and kept on ice. The transferred eluates were centrifuged for 10 minutes at 17000 rcf at RT in a suitable benchtop centrifuge to pellet the solid components. After centrifugation, the tubes were placed on ice under a class II safety cabinet and the supernatant was transferred into a new pre-cooled Eppendorf 1.5 ml Protein LoBind tube without pipetting solid or viscous portions. The volume of the transferred eluate was estimated. If blood stains could not be removed from the AM before centrifugation, the two AMs from one subject were eluted separately. In this case, the two eluates from one subject continue to be processed separately. The eluates were evaluated based on the available color scale (1-5) (FIG. 1). If a sample was rated color scale 5, the sample was not further processed.
Several working aliquots were prepared in pre-cooled Eppendorf 1.5 ml Protein LoBind tubes under a class II safety cabinet. Samples and aliquots were kept on ice during preparation. They were stored at −80 °C for long term storage.

### 2. Analytics of Nasal Fluid Samples

### 2.1 Measurement of Whole Protein Content

The total protein content of the nasal fluid sample (nasal secretion eluate) was measured by BCA Assay (Pierce BCA Protein Assay Kit, Thermo Fisher Scientific, 10678484). One sample aliquot was taken from the −80 °C freezer and equilibrated to RT on the bench for approximately 30 minutes. The total protein assay (BCA) was performed according to the manufacturer's protocol and the concentration was calculated in mg/mL. These results were further used for the normalization of the total protein concentration in gel electrophoresis in the following measurements.

### 2.2 Determination of Protein Specific Biomarker Signature for β Amyloid and Tau

The following steps describe the determination of the protein specific biomarker signature for the key analytes: β Amyloid 40 (Aβ40), β Amyloid 42 (Aβ42), total β Amyloid, phosphorylated Tau (p-Tau) and total Tau (t-Tau). The analysis was carried out using one or two antibodies against each analyte and using different capillary sizes (low, mid, high molecular size). Each measurement generates a signature that is specific to the respective marker protein and also specific for the individual disease state. The signature is basically made up of a large number of parameters. These parameters include the total level of analyte, a separation of the analyte by molecular weight based on its quaternary structures (e.g., monomers and different oligomers) and the level of the different quaternary structures (e.g., the level of monomers, the level of oligomers).

### 2.3 Determination of Aβ40

An aliquot of nasal fluid sample was used to determine the Aβ40 specific biomarker signature. The sample was run on an automated protein separation and immunodetection system (Simple Western™ Jess, BioTechne, 004-650) and, optionally on traditional gel electrophoresis and Western Blot (see protocol below). The aliquot was thawed at room temperature (RT) for 30 minutes. An appropriate amount of volume was taken of the sample so that the final total protein concentration in the assay was 1.25 mg/ml. The sample was diluted in sample buffer provided by the manufacturer. The diluted sample was loaded into the Simple Western™ Jess system and was started and run according to the manufacturer's instructions as single plex assay. Capillaries with gel percentages suitable for mid- (12 to 230 kDa Separation Module, 8×25 Capillary Cartridges, BioTechne, SM-W004). Primary antibody #1 (Human Amyloid β (aa1-40) Antibody, BioTechne/R&D, MAB96181-100) was used and is specific to the C-terminus of Aβ40. An enzyme-linked secondary antibody (Anti-Mouse Detection Module, BioTechne, DM-003) is then added followed by a chemiluminescent substrate. The Aβ40 biomarker signature was measured, displaying information on the total level of the analyte and the level of different molecular weight sizes (quaternary structure) of Aβ40 molecules within the same sample. As shown in FIGS. 2 and 4, this allowed to simultaneously detect mono- and oligomeric Aβ40 molecules and quantify them separately for each individual molecular weight, in total and/or in relation to each other. FIGS. 2 and 4 illustrate exemplary banding patterns of Aβ40 that can be measured in nasal secretion from patients with different clinical conditions. Each lane represents an individual patient. Along the x-axis the molecular weight is displayed. Larger Aβ40 quaternary structures, e.g., oligomers that have formed from aggregated monomers in the brain are not separated into monomers by SDS and, thus, have higher molecular weight. Each antibody-epitope recognition is represented by a chemiluminescent signal, thus higher signal intensities represent more antibody-epitope recognitions and thus represent higher amount of detected quaternary structures or overall levels of analyte. By calculating the area under the curve (AUC) the amount of antibody-epitope recognition can be measured and, thus, the level of a given quaternary structure or the total level of the protein can be back calculated. A graphical representation of the AUC, calculated from the chemiluminescent signal of the bound antibodies for Aβ40 can be seen in FIGS. 3 and 6 (for more information see Protein Simple—Jess user manual Revision G, December 2022; https://resources.bio-techne.com/bio-techne-assets/docs/software/Simple%20Western/Jess/Jess%20User%20Guide_Rev %20G.pdf). Oligomers of approximately 100 kDa, 56 kDa and 32 kDa, and 19 kDa can be detected, as well as monomers at approximately 4 kDa. As can be seen, for example, in FIG. 3 the 56 kDa oligomer band shows a higher signal in patients without AD (corresponding to a A− classification) in comparison to patients with clear AD (classified as A+). Each curve represents a single patient. The relative decrease in the 56 kDa band in patients with AD(A+) could be explained by the accumulation of Aβ40 in insoluble bigger Aβ42 aggregates within the brain. It is known that insoluble Aβ42 aggregates in the AD patients also sequester Aβ40 molecules leading to a decrease of detected Aβ-40. FIG. 4 shows an increase in the detected monomeric species (see* monomers FIG. 4) in patients without AD(A−) relative to patients with AD(A+). FIG. 5 shows the biomarker signature for Aβ40 from a patient with AD (A+) compared to a patient without AD (A−) which also demonstrates that higher levels of Aβ40 monomer at 4 kDa can be detected.

### 2.4 Determination of Aβ42 Specific Biomarker Signature

An aliquot of nasal secretion was used for the determination of the Aβ42 specific biomarker signature. The sample was run on an automated protein separation and immunodetection system (Simple Western™ Jess, BioTechne, 004-650), and optionally on traditional gel electrophoresis and Western Blot. The protocol described below refers to the automated system. The aliquot was thawed at room temperature for 30 minutes. An appropriate amount of volume was taken of the sample so that the final total protein concentration in the assay was 1.25 mg/ml. The sample was diluted in sample buffer provided by the manufacturer. The diluted sample was loaded into the Simple Western™ Jess system and the system was started and run according to the manufacturer's instructions as single plex assay. Capillaries with gel percentages suitable for mid- (12 to 230 kDa Separation Module, 8×25 Capillary Cartridges, BioTechne, SM-W004) molecular weight separation was used. Primary antibody #6 (Anti Amyloid β42(43), Monoclonal Antibody (BC05), Fujifilm Wako, 010-26903) was used and is specific to Aβ42 and may recognize one or several isoforms and oligomeric structures. An enzyme-linked secondary antibody (Anti-Mouse Detection Module, BioTechne, DM-003) is then added followed by a chemiluminescent substrate. The Aβ42 specific biomarker signature is generated, displaying information on the total amount of the analyte and the amount within different molecular weight sizes of Aβ42 molecules (quaternary structures) within the same sample. As shown in FIG. 6, this allowed to simultaneously detect different oligomeric Aβ42 molecules and quantify them separately for each individual size, in total and/or in relation to each other. FIG. 6 demonstrates two Aβ42 specific biomarker signatures, one is from a patient with AD (A+) in comparison to another from a patient without AD (A−). As evident from FIG. 6, oligomers of approximately 32 kDa, 56 kDa, 70 kDa and 92 kDa can be detected. The two samples, being run on a mid-molecular weight gel, show that i) the total amount/level of Aβ42 and ii) different (oligomeric) quaternary structures and their corresponding level can be measured. As demonstrated here, there is an overall decrease in the detected total level of Aβ42 (calculated by the total area under the curve for the whole biomarker signature) in an AD patient (classified as A+), relative to the total level of an A− individual. Also the two patients show altered band ratios between the 56 and 32 kDa oligomers. Further, as evident from FIG. 11, comparing the quantities of relative amount and the structures of the same marker protein in a nasal fluid sample and the CSF sample from the same individual, oligomeric bands of approximately 32 kDa, 56 kDa as well as further bands at approximately 75 kDa and 100 kDa can be detected in the nasal fluid sample that were not determinable in the corresponding CSF sample.

### 2.5 Determination of the Total Aβ Specific Biomarker Signature

An aliquot of nasal secretion was used for determination of the total Aβ specific biomarker signature. The sample was run on an automated protein separation and immunodetection system (Simple Western™ Jess, BioTechne, 004-650) and, optionally on traditional gel electrophoresis and Western Blot. The protocol described below refers to the automated system. The aliquot was thawed at room temperature for 30 minutes. An appropriate amount of volume was taken of the sample so that the final total protein concentration in the assay was 1.25 mg/ml. The sample was diluted in sample buffer provided by the manufacturer. The diluted sample was loaded into the Simple Western™ Jess system and the system was started and run according to the manufacturer's instructions as single plex assay. Capillaries with different gel percentages suitable for mid- (12 to 230 kDa Separation Module, 8×25 Capillary Cartridges, BioTechne, SM-W004) molecular weight separation was used. Primary antibody 6E10 (Purified anti-β-Amyloid, 1-16 Antibody (6E10), BioLegend, 803001) recognizes all forms of Aβ. An enzyme-linked secondary antibody (Anti-Mouse Detection Module, BioTechne, DM-003) is then added followed by a chemiluminescent substrate. The pan Aβ specific biomarker signature is generated, displaying information on the total level of the analyte, as well as the level of different molecular weight sizes of pan Aβ molecules (quaternary structures) within the same sample. As shown in FIG. 7, this allows to simultaneously detect mono- and oligomeric Aβ molecules and quantify them separately for each individual size, in total and in relation to each other. The pan-Aβ antibodies bind to an epitope that is more N-terminal than the species-specific antibodies described above. This allowed for recognition of different quaternary structures compared to the antibodies used in 3.2.1 and 3.2.2 above. FIG. 7 shows the pan-Aβ specific biomarker signature of a patient without Alzheimer's disease (AD). It is envisaged that in A+ vs A− individuals the ratio of the 45 and 32 kDa bands, the total levels, and/or the levels of higher oligomeric structures such as the 183 kDa band are altered.

### 2.6 Determination of Total Tau (t-Tau) Specific Biomarker Signature

An aliquot of nasal secretion was used for determination of the t-Tau specific biomarker signature. The sample was run on an automated protein separation and immunodetection system (Simple Western™ Jess, BioTechne, 004-650) and, optionally on traditional gel electrophoresis and Western Blot. The protocol described below refers to the automated system. The aliquot was thawed at room temperature for 30 minutes. An appropriate amount of volume was taken of the sample so that the final total protein concentration in the assay was 1.25 mg/ml. The sample was diluted in a sample buffer provided by the manufacturer. The diluted sample was loaded into the Simple Western™ Jess system and the system was started and run according to the manufacturer's instructions as single plex assay. Capillaries with different gel percentages suitable for low- (2 to 40 kDa Separation Module, 8×25 Capillary Cartridges, BioTechne, SM-W012), mid- (12 to 230 kDa Separation Module, 8×25 Capillary Cartridges, BioTechne, SM-W004), or high- (66 to 440 kDa Separation Module, 8×25 Capillary Cartridges, BioTechne, SM-W008) molecular weight separation were used. Primary antibody #7 (Purified anti-Tau, 404-441 Antibody (mouse), BioLegend, 806601) was used, and is specific to t-Tau and recognize one or several isoforms and oligomeric structures. An enzyme-linked secondary antibody (Anti-Mouse Detection Module, BioTechne, DM-003) is then added followed by a chemiluminescent substrate. The t-Tau specific biomarker signature is generated, displaying information on the total level of the analyte, as well as the level of quaternary structures with different molecular weight within the same sample. As shown in FIG. 8, this allowed to simultaneously detect mono- and oligomeric t-Tau molecules and quantify them separately for each individual molecular weight, in total and/or in relation to each other. The canonical isoform of t-Tau, seen at approximately 55 kDa is detected at a higher level in patients with clear signs for neurodegeneration (classified as N+) than in patients without signs for neurodegeneration (classified as N−) (FIG. 8). Different other quaternary structures can be measured as well, e.g. total Tau dimers. The ratio of t-Tau dimers to monomers also can be used to compute the classification specific score for N classification (FIG. 8). Further, as evident from FIG. 12, when comparing the quantities of the relative amount and the structures of the same marker protein tTau in a nasal fluid sample and a CSF sample from the same individual (Note: the experiment was done with a samples from another subject than reported in FIG. 8), oligomeric bands of approximately 63 kDa can be detected in both samples from the same individual with nearly identical intensities. Since it was 6 times more CSF sample volume applied then corresponding nasal fluid, this is indirectly indicating that the amount of structures seen at 63 kDa in the nasal secretion sample is at least comparable to CSF or higher than in CSF.

### 2.7 Determination of p-Tau-181 Specific Biomarker Signature

An aliquot of nasal secretion was used to determine the p-Tau-181 specific biomarker signature. The sample was run on an automated protein separation and immunodetection system (Simple Western™ Jess, BioTechne, 004-650) and, optionally on traditional gel electrophoresis and Western Blot. The protocol described below refers to the automated system. The aliquot was thawed at room temperature for 30 minutes. An appropriate amount of volume was taken of the sample so that the final total protein concentration in the assay was 1.25 mg/ml. The sample was diluted in sample buffer provided by the manufacturer. The diluted sample was loaded into the Simple Western™ Jess system and the system was started and run according to the manufacturer's instructions as single plex assay. Capillaries with different gel percentages suitable for low- (2 to 40 kDa Separation Module, 8×25 Capillary Cartridges, BioTechne, SM-W012), mid- (12 to 230 kDa Separation Module, 8×25 Capillary Cartridges, BioTechne, SM-W004), or high- (66 to 440 kDa Separation Module, 8×25 Capillary Cartridges, BioTechne, SM-W008) molecular weight separation were used. Primary antibody #8 (Phospho-Tau (Thr181) (D9F4G) Rabbit mAb, Cell Signaling, 12885S) is specific to pTau-181 and recognizes one or several isoforms and oligomeric structures. An enzyme-linked secondary (Anti-Rabbit Detection Module, BioTechne, DM-001) is then added followed by a chemiluminescent substrate. The pTau-181 specific biomarker signature is measured, displaying information on the total level of the analyte and the level of quaternary structures with different molecular weight within the same sample. As shown in FIG. 9, this allowed us to simultaneously detect mono- and oligomeric pTau-181 molecules and quantify them separately for each individual molecular weight, in total and/or in relation to each other. As shown in FIG. 9, the total level of p-Tau-181 can be calculated by the area under the curve (AUC). As shown here, in this individual the total level of detected p-Tau is decreased in patients with clear Tau pathology (T+) compared to patients without Tau pathology (T−). Also shown here, a decreased level of monomers, present at approximately 56 kDa as well as decreased levels of dimers and trimers with apparent molecular sizes at 96 and 139 kDa, respectively can be seen in patients with clear tau pathology (T+). Further, analyzing the corresponding samples of another individual (Note: the experiment was done with a samples from another subject than reported in FIG. 9), as evident from FIG. 13, and when comparing the quantities of the relative amount and the structures of the same marker protein pTau in a nasal fluid sample and a CSF sample from the same individual, oligomeric bands of approximately 60 kDa, 100 kDa, 146 kDa and 230 kDa can be detected in both samples from the same individual with nearly identical intensities. Since it was 6 times more CSF sample volume applied then corresponding nasal fluid volume, this is indirectly indicating that the amount of structures seen at 60 kDa, 100 kDa, 146 kDa and 230 kDa in the nasal secretion sample are at least comparable to CSF or higher than in corresponding CSF from the same individual.

### 3. Generation of CSF Reference Data

### 3.1 Comparison of Corresponding CSF and Nasal Fluid Core Marker Pattern

In order to compare the nasal fluid based measurements of core markers to CSF based measurements of the same core markers, nasal fluid samples and CSF samples were collected from the same individuals, resulting in corresponding samples.
Within these corresponding samples the intention was
i) to demonstrate the quantities as well as inter-protein and intra-protein correlations (the pattern (signature) of the protein(s)) of the core AD marker protein(s);
ii) to analyze the corresponding samples regarding the distribution and the quantities of their protein specific monomeric and oligomeric structure(s) of the AD marker protein(s) in the nasal fluid sample as well as in a corresponding CSF sample obtained from the same subject. Unexpectedly, it was found that the signature of the AD marker protein(s) Aβ40, tTau and pTau181 are nearly identical by a direct comparison. Notably, it was found that the signature of Aβ42 marker protein differs in the nasal fluid when compared to a corresponding CSF sample obtained from the same subject;
iii) the distribution pattern of the protein specific monomeric- and oligomeric structures as well as their respective quantities within the two corresponding bodily fluids; and
iv) to demonstrate neurodegenerative disease specific differences regarding relative protein amounts of the marker protein(s), and the distribution of monomeric- and oligomeric structures of the marker protein(s) within different subjects with for example an underlying amyloid pathology, compared to subjects without amyloid pathology.
Side note: AD was chosen as a representative for all neurodegenerative diseases that can be assessed through analysis of brain derived markers because
i) it is the most common one and therefore a tremendous amount of data and knowledge exist;
ii) there are multiple markers whose quantities can up to date be reliably distinguished in CSF through in vitro diagnostic methods; and
iii) the AD specific inter-protein and intra-protein correlations in CSF, the protein pattern, has been evaluated in broad studies worldwide and the level of confidence for the trueness is high throughout the globe.

### 3.2 CSF Collection

CSF samples were also obtained from the subjects from whom the nasal fluid samples were obtained (see sections 1 to 3, supra). The CSF sample was collected at the clinical site by lumbar puncture, preferably in the morning and was performed between the 3rd to 4th or 4th to 5th lumbar vertebrae. It was collected in a polypropylene collection tube, discarding the first 20 drops, following international guidelines. Too much empty space in the tube was avoided. Within the next 4 hours after collection, the CSF was centrifuged for 10 minutes at approximately 2000 rcf, at room temperature (RT) and transferred into a new polypropylene collection tube. 1500 μl of the transferred CSF were aliquoted into a separate polypropylene collection tube. CSF samples and aliquots were stored in a suitable box at −80 °C within 4 hours after collection.

### 3.3 CSF Specimen Transport

The box containing the CSF aliquots and a temperature logger was transferred into a styrofoam box with the lid facing up. The whole styrofoam box was filled with dry ice. The sample box was completely surrounded by the dry ice. Samples are then shipped over night to the analytical lab by a commercial logistics partner.

### 3.4 Sample Handling and Pre-Analytics

### 3.4.1 Sample Receipt, Inspection, and Storage

The styrofoam box was opened at the analytical lab shortly after delivery. It was checked if the collected samples were in good condition. This means, that they were frozen at −80 °C during the whole shipment (checked by the status of the remaining dry ice and the report of the included temperature logger) and the integrity of the tubes was fine (unbroken, sealed). The samples were then stored at the analytical lab in specific racks or boxes in the −80 °C freezer until preprocessing.

### 3.4.2 Sample Measurement for CSF

The CSF aliquot was thawed, and measurement was performed using Immunoassay-based measurements, e.g. on a Fuji Rebio Lumipulse platform, according to the manufacturer's protocol for all core analytes: Amyloid-β40, Amyloid-β42, pTau181 and tTau.

### 3.4.3 Sample Measurement for Nasal Fluid Sample

In order to evaluate and compare the marker proteins in a summary-evaluation in the sense of an integrated overall result and to be able to compare pattern of the marker proteins in between the two body fluids, the correlations of the marker proteins, were analyzed for these evaluations using a comparable measurement technology.
Since, as described above, in vitro analysis of CSF for the analysis of the AD marker proteins was done using immunoassays, nasal fluid sample analysis of the AD marker proteins was also done using a semi-automated immunoassay (See FIGS. 14 to 17).
Nasal fluid samples were analyzed using the following SIMOA-kits for all core AD analytes on a SR-X Instrument (Quanterix) and following the manufacturer's instructions.
   SIMOA pTau-181 Advantage V2 Kit (Quanterix, 103714) in a 1:5 dilution
   SIMOA Tau Advantage Kit (Quanterix, 101552) in a 1:20 dilution
   SIMOA Aß-40 Advantage Kit (Quanterix, 101672) in a 1:5 dilution
   SIMOA Aß-42 Advantage Kit (Quanterix, 101664) in a 1:2.5 dilution.

### 4. Data Processing for Immunoassay-Based Measurements

CSF and nasal fluid sample-based measurement results of all four analytes were used for subsequent data analysis.
Corresponding data sets were stored in Excel files and processed with Python scripts using standard libraries such as pandas and NumPy. Linear and logistic regression were calculated using the sklearn package. Graphics were generated using the matplotlib and seaborn packages. For quantitative analysis, primarily these methods were used to draw conclusions from the data. P-value for the probability of measuring the same or larger difference between the observed samples if the underlying distribution is indeed the same; smaller values indicate a significant difference. Values of p<0.05 are described as significant. We also verified that non-parametric alternatives to Student's t (Mann-Whitney, Welch) lead to similar values and conclusions.
When comparing two variables (e.g., different marker/analytes) from the same cohort, we used linear regression. The outcome (besides a fitted line giving the optimal linear relationship between the quantities) is Pearson's “r”, that is a parameter for the goodness of fit, and Pearson's p is a parameter for the statistical significance. The parameter “r” indicates to what degree one of the quantities is determined by the other, with a range of 0.1 to 0.3 counting as a weak correlation, 0.3 to 0.5 as moderate correlation, and 0.5 and more as strong correlation. The significance p again indicates the probability of measuring such a degree of correlation or more by chance if the quantities were independent.

### 4.1 Data Processing of Protein-Specific Biomarker Signatures

The corresponding nasal fluid and CSF samples were the additionally run on an automated protein separation and immunodetection system (Simple Western™ Jess, BioTechne, 004-650), and optionally on traditional gel electrophoresis and Western Blot to demonstrate the nearly identical distribution of protein specific monomeric- and oligomeric structures as well as to demonstrate the respective quantities within these different quaternary structures and the relations thereof. The protocols described refer to the automated system. The nasal fluid aliquots were initially thawed at room temperature for 30 minutes. An appropriate amount of volume was taken of each of the nasal fluid samples so that the final total protein concentration in the assay was 1.25 mg/ml. The sample was diluted in sample buffer provided by the manufacturer. The diluted sample was loaded into the Simple Western™ Jess system and the system was started and run according to the manufacturer's instructions as single plex assay. Capillaries with different gel percentages suitable for low- (2 to 40 kDa Separation Module, 8×25 Capillary Cartridges, BioTechne, SM-W012), mid- (12 to 230 kDa Separation Module, 8×25 Capillary Cartridges, BioTechne, SM-W004), or high- (66 to 440 kDa Separation Module, 8×25 Capillary Cartridges, BioTechne, SM-W008) molecular weight separation were used. Primary antibodies specific to the core AD marker proteins were used that recognizes one or several marker protein specific isoforms and oligomeric structures. An enzyme-linked secondary antibody is then added followed by a chemiluminescent substrate. The marker protein specific biomarker signature is measured, displaying information on i) the total level of the analyte and ii) the maximum position as well as the quantities of quaternary structures at different molecular weight within the same sample. As shown in FIGS. 10, 11, 12 and 13 this allows to simultaneously detect mono- and oligomeric molecules and quantify them separately for each individual molecular weight, in total and/or in relation to each other.
On the same measurement platform corresponding CSF samples from the same individuals/subjects were applied according to the above-mentioned protocol with the only exception that 6 times the volume of CSF was applied and compared to the applied volume of the nasal fluid samples. As previously described CSF was collected in the lumbar area. In CSF the core protein marker for AD gets diluted in a large volume, therefore the fluid itself is not very protein rich.
Each measurement of a corresponding body fluid sample set on the automated protein separation and immunodetection system yielded in a set of data for each marker protein. As shown in FIGS. 10, 11, 12 and 13, the measurements are displayed in a spectrum that gives an intensity (corresponding to the concentration of protein-antibody complexes, displayed on the y-axis) as a function of the molecular weight (monomeric or oligomeric structures are displayed on the x-axis). A software provides a way to apply a baseline correction algorithm to the spectra, and the corrected spectra are used as displayed in FIGS. 3, 5, 6, 7, 8, 9, 10, 11, 12 and 13.
By comparing the total intensity in the generated spectrum by numerically integrating the intensity over the molecular weight in the quantifiable range—i.e., calculating the area under the curve (AUC) with the correct weighting, one can also calculate the contribution to the signal from individual bands or groups of bands by applying a fit algorithm that fits a set of peaks.
The result for each sample and run is a set of measurements for one analyte, which we call the protein-specific biomarker signature and which includes, but is not limited to, the total intensity and the intensity from each of the displayed bands.
So besides the intensities of each band, which is equal to the quantities of each molecular size, the distribution of these bands within a certain molecular range can be seen. Additionally, this allows to evaluate ratios or relations of different band sizes and quantities towards each other.
Since similarities and or differences between the two body fluids can be seen by eye here, no additional data are provided here to compare corresponding nasal fluid and CSF based measurements.

1. A nasal fluid sample obtained from a subject, wherein the nasal fluid sample comprises the marker proteins β amyloid (Aβ), phosphorylated Tau (pTau) and/or total Tau (tTau).

2. The nasal fluid sample of claim 1, wherein the nasal fluid sample is a purified nasal fluid sample.

3. The nasal fluid sample of claim 1 or 2, wherein the concentration of the marker protein(s) is (are) equivalent or at least 1.1-fold increased compared to a cerebrospinal fluid (CSF) sample obtained from the same subject.

4. The nasal fluid sample of any one of claims 1 to 3, wherein the nasal fluid sample is characterized by a pTau protein concentration that is smaller than the tTau protein concentration.

5. The nasal fluid sample of any one of claims 1 to 4, wherein the relative amount of Aβ42/Aβ40 is greater than 0 and less than 1.

6. The nasal fluid sample of any one of claims 1 to 5, wherein the Aβ marker is characterised by a molecular weight of about 4 kDa, about 8 kDa, about 12 kDa, about 16 kDa, about 19 kDa, about 24 kDa, about 32 kDa, about 40 kDa, about 44 kDa, about 48 kDa, about 52 kDa, about 56 kDa, about 60 to 72 kDa, about 84 to 120 kDa and/or more than about 140 kDa.

7. The nasal fluid sample of any one of claims 1 to 5 wherein the pTau marker is characterised by a molecular weight of about 20 kDa, about 30 kDa, about 38 kDa, about 55 to 62 kDa, about 96 to 106 kDa, and about 140 to 160 kDa and/or more than about 180 kDa.

8. The nasal fluid sample of any one of claims 1 to 5 wherein tTau marker is characterised by a molecular weight of about 30 kDa, about 38 kDa, about 48 kDa, about 55 to 62 kDa, about 96 to 106 kDa, about 140 to 160 kDa and more than 160 kDa.

9. Use of the nasal fluid sample of any one of claims 1 to 8 for the aid in diagnosis of a neurodegenerative disease.

10. A method for the aid in diagnosis of a neurodegenerative disease of a subject in a nasal fluid sample, wherein the method comprises the steps of:
(a) determining the protein concentration of the marker proteins phosphorylated Tau (pTau), total Tau (tTau) and/or, optionally β amyloid (Aβ), in the nasal fluid sample as defined in any one of claims 1 to 8;
(b) comparing the protein concentrations of the marker proteins as determined in the nasal fluid sample with the protein concentration in controls; and
(c) determining whether the values of the marker proteins are pathologically altered in relation to the protein concentration of controls.

11. The method of claim 10, wherein the neurodegenerative disease is selected from the group consisting of Alzheimer's disease (AD), Parkinson's disease (PD), chronic traumatic encephalopathy (CTE), frontotemporal dementia (FTD), Pick's disease and Creutzfeldt-Jakob's disease (CJD).

12. The method of claim 10 or 11, wherein the relative amount of Aβ42/Aβ40 which correlates negatively with pTau and/or tTau is evidence of a neurodegenerative disease in a subject.

13. The method of claim 10 or 11, wherein the relative amount of Aβ42/Aβ40 which correlates positively with pTau and/or tTau is evidence of a neurodegenerative disease in a subject.

14. The method of claim 12, wherein any one of (i) or (ii) applies:
(i) pTau correlates positively with tTau and Aβ42 correlates positively with Aβ40; and
(ii) the relative amount of Aβ42/Aβ40 is greater than 0 and less than 1.

15. The method of item 13, wherein any one of (i) or (ii) applies:
(i) pTau correlates positively with Aβ42/Aβ40 and Aβ42 correlates negatively with Aβ40; and
(ii) the relative amount of Aβ42/Aβ40 is greater than 0 and less than 1.

16. The method of any one of claims 10 to 15, wherein the neurodegenerative disease is AD.

17. The use of the nasal fluid sample of claim 9, wherein the neurodegenerative disease is selected from the group consisting of Alzheimer's disease (AD), Parkinson's disease, chronic traumatic encephalopathy (CTE), frontotemporal dementia (FTD), Pick's disease and Creutzfeldt-Jakob's disease (CJD).
